(19) United States
(12) Patent Application Publication
(10) Pub. No.: US 2019/0055603 A1
(43) Pub. Date: Feb. 21, 2019

(54) SYSTEM AND METHOD FOR PROCESSING GENOTYPE INFORMATION RELATING TO DRUG METABOLISM

(71) Applicant: PROOVE BIOSCIENCES, INC., Annapolis, MD (US)

(72) Inventor: Brian MESHKIN, Ladera Ranch, CA (US)

(21) Appl. No.: 15/570,311

(22) PCT Filed: Apr. 28, 2016
(86) PCT No.: PCT/US2016/029900

§ 371 Date: Oct. 27, 2017

Related U.S. Application Data
(60) Provisional application No. 62/153,755, filed on Apr. 28, 2015.

(51) Int. Cl. C12Q 1/6883; G16H 50/20; C12Q 1/02; G06F 19/10; G16H 10/60

(52) U.S. Cl. CPC C12Q 1/6883; G16H 50/20; C12Q 1/025; C12Q 2600/106; G16H 10/60; C12Q 2600/156; C12Q 2600/172; G06F 19/10

(57) ABSTRACT

There are systems and methods for preparing or using prognostic information about drug metabolism response. The information may include determining patient information, including DNA information, associated with a human subject; determining from the DNA information whether a subject genotype of the human subject includes one or more alleles or haplotypes in the one or more genes of the subject genotype by detecting, utilizing a detection technology and the DNA information, a presence or absence of the one or more alleles or haplotypes.

### PRIORITY

This application claims priority to U.S. Provisional Application No. 62/153,755 entitled “System and Method for Processing Genotype Information Relating to Drug Metabolism” by Brian Meshkin filed on Apr. 28, 2015, which is incorporated herein by reference in its entirety.

### BACKGROUND

In nature, organisms of the same species usually differ from each other in various aspects such as in their appearance or in one or more aspects of their biology. The differences are often based on genetic distinctions, some of which are called polymorphisms. Polymorphisms are often observed at the level of the whole individual (i.e., phenotype polymorphism), in variant forms of proteins and blood group substances (i.e., biochemical polymorphism), in morphological features of chromosomes (i.e., chromosomal polymorphism), and at the level of DNA in differences of nucleotides and/or nucleotide sequences (i.e., genetic polymorphism).

Examples of genetic polymorphisms include alleles and haplotypes. An allele is an alternative form of a gene, such as one member of a pair, that is located at a specific position on a chromosome and are known as single nucleotide polymorphisms (SNPs). A haplotype is a combination of alleles, or a combination of SNPs on the same chromosome. An example of a genetic polymorphism is an occurrence of one or more genetically alternative phenotypes in a subject due to the presence or absence of an allele or haplotype.

Genetic polymorphisms can play a role in determining differences in an individual's response to a species of drug, a drug dosage or a therapy including one drug or a combination of drugs. Pharmacogenetics and pharmacogenomics are multidisciplinary research efforts to study the relationships among genotypes, gene expression profiles, and phenotypes, as often expressed through the variability between individuals in response to the drugs taken. Since the initial sequencing of the human genome, more than a million SNPs have been identified. Some of these SNPs have been used to predict clinical predispositions or responses based upon data gathered from pharmacogenomic studies.

A patient's genotype information is often utilized to help a prescriber decide between medications based on information associated with a patient's genetic profile (i.e., genotype information). There is a desire to utilize a patient's genotype information in determining the patient's predisposition to drug metabolism of individual drugs and drug panels. There is also a desire for methods for predicting and/or diagnosing individuals exhibiting irregular drug metabolism of individual drugs and drug panels. Furthermore, there is also a desire to determine genetic information, such as polymorphisms, which may be utilized for predicting variations in predisposition to metabolism of individual drugs and drug panels. There is also a desire to implement systems processing and distribing the detected genetic information in a systematic way. Such genetic information would be useful in providing prognostic information about treatment options for a patient based on the patient's metabolism of individual drugs and drug panels.

Although it is known generally that drug metabolism may be associated with genetics—a factor not routinely considered, there is no rigourous methodology to systematically provide doctors with an ability to identify patients exhibiting irregular drug metabolism of individual drugs and drug panels. Such systems and methods would be beneficial to provide information that improves accuracy in identifying patients at risk for irregular drug metabolism.

Given the foregoing, and to address the above-described limitations, systems and methods are desired for identifying, estimating and/or determining a potential for success of an individual patient's clinical outcome in response to being treated with one or more drugs based on the patient's metabolism.

### SUMMARY

This summary is provided to introduce a selection of concepts that are further described in the Detailed Description below. The genes, polymorphisms, sequences and sequence identifiers (i.e., SEQ IDs or SEQ ID Numbers) listed or referenced herein are also described in greater detail below in the Detailed Description. This summary is not intended to identify key or essential features of the claimed subject matter. Also, this summary is not intended as an aid in determining the scope of the claimed subject matter.

The present invention meets the above-identified needs by providing systems, methods and computer readable mediums (CRMs) for preparing and utilizing prognostic information associated with a predisposition to risk for irregular drug metabolism in a patient. The prognostic information is derived from genotype information about a patient's gene profile. The genotype information may be obtained by, inter alia, assaying a sample of genetic material associated with a patient.

The systems, methods and CRMs, according to the principles of the invention, can be utilized to determine prognostic information associated with drug metabolism in a patient. The prognostic information may be used for addressing prescription needs directed to caring for an individual patient. It may also be utilized in managing large healthcare entities, such as insurance providers, utilizing comprehensive business intelligence systems. These and other objects are accomplished by systems, methods and CRMs directed to preparing and utilizing prognostic information associated with drug metabolism in a patient, in accordance with the principles of the invention.

According to a first principal of the invention, there is a method. The method may include facilitating a processing of and/or processing (1) data and/or (2) information and/or (3) at least one signal, the (1) data and/or (2) information and/or (3) at least one signal based, at least in part, on any combination of at least part of the following: determining patient information, including DNA information, associated with a human subject; determining from the DNA information whether a subject genotype of the human subject includes one or more alleles or haplotypes in at least one gene by detecting, utilizing a detection technology and the DNA information, a presence or absence of the one or more alleles or haplotypes in the one or more genes of the subject genotype, wherein the at least one gene is selected from the gene group: CYP2C8, CYP2C9, CYP2C19, CYP3A4, CYP3A5, CYP2D6, CYP1A2, VKORC1, UGT2B7, CYP2B6 and CYP2E1; determining at least one allele score associated with the alleles or haplotypes in the one or more genes based on the detected presence or absence of the one or more alleles or haplotypes in the one or more genes; determining at least one grade associated with one or more genes of the at least one gene based on the determined at least one allele score; and determining a drug metabolism response by the human subject associated with at least one drug based on the determined at least one grade associated with the one or more genes of the at least one gene.

The method may also include wherein the (1) data and/or (2) information and/or (3) at least one signal are further based, at least in part, on any combination of the following: determining at least one drug dosage recommendation based on the determined drug metabolism response associated with the at least one drug, wherein the method for determining the drug metabolism response associated with the human subject, is an ex vivo method; determining a comparing of a region, including the one or more alleles or haplotypes, of the subject genotype with a corresponding region of a predetermined reference genotype, wherein characteristics of the corresponding region of the reference genotype are based upon a predetermined population norm; determining prognostic information associated with the human subject based on the determined drug metabolism response; and determining a therapy for the human subject based on the determined prognostic information associated with the human subject, wherein the at least one gene includes at least any number from two to eleven genes selected from the gene group.

According to a second principal of the invention, there is an apparatus. The apparatus may include any combination of at least one processor; and at least one memory including computer program code for one or more programs, the at least one memory and the computer program code configured to, with the at least one processor, cause the apparatus to perform at least the following, determine patient information, including DNA information, associated with a human subject; determine from the DNA information whether a subject genotype of the human subject includes one or more alleles or haplotypes in at least one gene by detecting, utilizing a detection technology and the DNA information, a presence or absence of the one or more alleles or haplotypes in the one or more genes of the subject genotype, wherein the at least one gene is selected from the gene group: CYP2C8, CYP2C9, CYP2C19, CYP3A4, CYP3A5, CYP2D6, CYP1A2, VKORC1, UGT2B7, CYP2B6 and CYP2E1; determine at least one allele score associated with the alleles or haplotypes in the one or more genes based on the detected presence or absence of the one or more alleles or haplotypes in the one or more genes; determine at least one grade associated with one or more genes of the at least one gene based on the determined at least one allele score; and determine a drug metabolism response by the human subject associated with at least one drug based on the determined at least one grade associated with the one or more genes of the at least one gene.

According to a third principal of the invention, there is a non-transitory computer readable medium. The medium may store any combination of computer readable instructions that when executed by at least one processor perform a method, the method comprising facilitating a processing of and/or processing (1) data and/or (2) information and/or (3) at least one signal, the (1) data and/or (2) information and/or (3) at least one signal based, at least in part, on any combination of the following: determining patient information, including DNA information, associated with a human subject; determining from the DNA information whether a subject genotype of the human subject includes one or more alleles or haplotypes in at least one gene by detecting, utilizing a detection technology and the DNA information, a presence or absence of the one or more alleles or haplotypes in the one or more genes of the subject genotype, wherein the at least one gene is selected from the gene group: CYP2C8, CYP2C9, CYP2C19, CYP3A4, CYP3A5, CYP2D6, CYP1A2, VKORC1, UGT2B7, CYP2B6 and CYP2E1; determining at least one allele score associated with the alleles or haplotypes in the one or more genes based on the detected presence or absence of the one or more alleles or haplotypes in the one or more genes; determining at least one grade associated with one or more genes of the at least one gene based on the determined at least one allele score; and determining a drug metabolism response by the human subject associated with at least one drug based on the determined at least one grade associated with the one or more genes of the at least one gene.

The above summary is not intended to describe each embodiment or every implementation of the present invention. Further features, their nature and various advantages are made more apparent from the accompanying drawings and the following examples and embodiments.

### BRIEF DESCRIPTION OF THE DRAWINGS

Features and advantages of the present invention become more apparent from the detailed description, set forth below, when taken in conjunction with the drawings. In the drawings, like reference numbers indicate identical or functionally similar elements. Additionally, a left-most digit of a reference number identifies a drawing in which the reference number first appears. In addition, it should be understood that the drawings in the figures which highlight an aspect, methodology, functionality and/or advantage of the present invention, are presented for example purposes only. The present invention is sufficiently flexible such that it may be implemented in ways other than shown in the accompanying figures.

FIG. 1 is a block diagram illustrating an assay system which may be utilized for preparing genotype information from a sample of genetic material, according to an example;

FIG. 2 is a block diagram illustrating a prognostic information system which may be utilized for preparing and/or utilizing prognostic information utilizing the genotype information prepared using the assay system of FIG. 1, according to an example;

FIG. 3 is a flow diagram illustrating a prognostic information process for identifying a risk to a patient utilizing the assay system of FIG. 1 and the prognostic information system of FIG. 2, according to an example; and

FIG. 4 is a block diagram illustrating a computer system providing a platform for the assay system of FIG. 1 or the prognostic information system of FIG. 2, according to various examples.

### DETAILED DESCRIPTION

The present invention is useful for preparing and/or utilizing prognostic information about a patient. The prognostic information may be utilized to determine an appropriate therapy for the patient based on their genotype and phenotype information to identify their genetic predisposition to drug metabolim risk. The genetic predisposition may be associated with the selection of an individual drug or a combination of drugs, a dosage of the medication(s) and the utilization of the medication(s) in a regimen for treating the patient's medical condition.

The prognostic information may also be utilized for determining dose adjustments that may help a prescriber understand why a patient is or is not responding to a medication dosage, such as an “average” dose. The prognostic information may also be utilized by a prescriber to decide between medications based on the patient's genetic predisposition to drug metabolism risk to various drugs.

The prognostic information may also be utilized for predicting and/or diagnosing individuals exhibiting a regular or irregular predisposition to drug metabolism risk. Such genetic information can be very useful in providing prognostic information about treatment options for a patient. The patient may be associated with a medical condition. The patient may also have already been prescribed a medication for treating the medical condition. The present invention has been found to be advantageous for determining a treatment for a patient who may have a regular or irregular predisposition to drug metabolism risk. While the present invention is not necessarily limited to such applications, various aspects of the invention may be appreciated through a discussion of the various examples in this context, as illustrated through the examples below.

For simplicity and illustrative purposes, the present invention is described by referring mainly to embodiments, principles and examples thereof. In the following description, numerous specific details are set forth in order to provide a thorough understanding of the examples. It is readily apparent however, that the embodiments may be practiced without limitation to these specific details. In other instances, some embodiments have not been described in detail so as not to unnecessarily obscure the description. Furthermore, different embodiments are described below. The embodiments may be used or performed together in different combinations.

The operation and effects of certain embodiments can be more fully appreciated from the examples described below. The embodiments on which these examples are based are representative only. The selection of embodiments is to illustrate the principles of the invention and does not indicate that variables, functions, conditions, techniques, configurations and designs, etc., which are not described in the examples are not suitable for use, or that subject matter not described in the examples is excluded from the scope of the appended claims and their equivalents. The significance of the examples can be better understood by comparing the results obtained therefrom with potential results which can be obtained from tests or trials that may be or may have been designed to serve as controlled experiments and provide a basis for comparison.

Before the systems and methods are described, it is understood that the invention is not limited to the particular methodologies, protocols, systems, platforms, assays, and the like which are described, as these may vary. It is also to be understood that the terminology used herein is intended to describe particular embodiments of the present invention, and is in no way intended to limit the scope of the present invention as set forth in the appended claims and their equivalents.

Throughout this disclosure, various publications, such as patents and published patent specifications, are referenced by an identifying citation. The disclosures of these publications are hereby incorporated by reference in their entirety into the present disclosure in order to more fully describe the state of the art to which the invention pertains.

The practice of the present invention employs, unless otherwise indicated, conventional techniques of molecular biology, microbiology, cell biology, biochemistry and immunology, which are within the skill of the art. Such techniques are explained fully in the literature for example in the following publications. See, e.g., Sambrook and Russell eds. MOLECULAR CLONING: A LABORATORY MANUAL, 3rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5th edition (2005)); OLIGONUCLEOTIDE SYNTHESIS (M. J. Gait ed. (1984)); Mullis et al., U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)); MANIPULATING THE MOUSE EMBRYO: A LABORATORY MANUAL 3rd edition (Cold Spring Harbor Laboratory Press, Cold Spring Harbor, N.Y. (2002)). DEFINITIONS.

As used herein, certain terms have the following defined meanings. As used herein, the singular form “a,” “an” and “the” includes the singular and plural references unless the context clearly dictates otherwise. For example, the term “a cell” includes a single cell and a plurality of cells, including mixtures thereof.

As used herein, the terms “based on,” “comprises,” “comprising,” “includes,” “including,” “has,” “having” or any other variation thereof, are intended to cover a non-exclusive inclusion. For example, a system, process, method, article, or apparatus that comprises a list of elements is not necessarily limited to only those elements but may include other elements not expressly listed or inherent to such system, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B is true (or present).

All numerical designations, e.g., pH, temperature, time, concentration, and molecular weight, including ranges, are approximations which may be varied (+) or (−) by minor increments, such as, of 0.1. It is to be understood, although not always explicitly stated, that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known to those of ordinary skill in the art.

The term “allele” which is used interchangeably herein with the term “allelic variant” refers to alternative forms of a gene or any portions thereof. Alleles may occupy the same locus or position on homologous chromosomes. When a subject has two identical alleles of a gene, the subject is said to be homozygous for the gene or allele. When a subject has two different alleles of a gene, the subject is said to be heterozygous for the gene or allele. Alleles of a specific gene can differ from each other in a single nucleotide, or several nucleotides, and can include substitutions, deletions and insertions of nucleotides. An allele of a gene can also be an ancestral form of a gene or a form of a gene containing a mutation.

The term “haplotype” refers to a combination of alleles on a chromosome or a combination of SNPs within an allele on one chromosome. The alleles or SNPs may or may not be at adjacent locations (loci) on a chromosome. A haplotype may be at one locus, at several loci or an entire chromosome.

The term “ancestral,” when applied to describe an allele in a human, refers to an allele of a gene that is the same or nearest to a corresponding allele appearing in the corresponding gene of the chimpanzee genome. Often, but not always, a human ancestral allele is the most prevalent human allelic variant appearing in nature—i.e., the allele with the highest gene frequency in a population of the human species.

The term “wild-type,” when applied to describe an allele, refers to an allele of a gene which, when it is present in two copies in a subject, results in a wild-type phenotype. There can be several different wild-type alleles of a specific gene. Also, nucleotide changes in a gene may not affect the phenotype of a subject having two copies of the gene with the nucleotide changes.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region may include, for example, a single nucleotide polymorphism (SNP), the identity of which differs in the different alleles by a single nucleotide at a locus in the polymorphic region of the gene. In another example, a polymorphic region may include a deletion or substitution of one or more nucleotides at a locus in the polymorphic region of the gene.

The expression “amplification of polynucleotides” includes methods such as PCR, ligation amplification (or ligase chain reaction, LCR) and other amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, a PCR procedure is a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e., each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively, the amplified sequence(s) may be cloned prior to sequence analysis. Methods for direct cloning and sequence analysis of enzymatically amplified genomic segments are known in the art.

The term “encode,” as it is applied to polynucleotides, refers to a polynucleotide which is said to “encode” a polypeptide. The polynucleotide is transcribed to produce mRNA, which is then translated into the polypeptide and/or a fragment thereof by cell machinery. An antisense strand is the complement of such a polynucleotide, and the encoding sequence can be deduced therefrom.

As used herein, the term “gene” or “recombinant gene” refers to a nucleic acid molecule comprising an open reading frame and including at least one exon and optionally an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. A “related” or “homologous” sequence shares identity with a comparative sequence, such as 100%, at least 99%, at least 95%, at least 90%, at least 80%, at least 70%, at least 60%, at least 50%, at least 40%, at least 30%, at least 20%, or at least 10%. An “unrelated” or “non-homologous” sequence shares less identity with a comparative sequence, such as less than 95%, less than 90%, less than 80%, less than 70%, less than 60%, less than 50%, less than 40%, less than 30%, less than 20%, or less than 10%.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof.

The term “isolated” as used herein with respect to nucleic acids, such as DNA or RNA, refers to molecules separated from other DNAs or RNAs, respectively, which are present in a natural source of a macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

As used herein, the term “nucleic acid” refers to polynucleotides such as deoxyribonucleic acid (DNA), and, where appropriate, ribonucleic acid (RNA). The term “nucleic acid” should also be understood to include, as equivalents, derivatives, variants and analogs of either RNA or DNA made from nucleotide analogs, and, as applicable to the embodiment being described, single (sense or antisense) and double-stranded polynucleotides.

Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine,” “cytidine,” “guanosine,” and “thymidine” are used. It is understood that if the nucleic acid is RNA, it includes nucleotide(s) having a uracil base that is uridine.

The terms “oligonucleotide” or “polynucleotide,” or “portion,” or “segment” thereof refer to a stretch of polynucleotide residues which may be long enough to use in PCR or various hybridization procedures to identify or amplify identical or related parts of mRNA or DNA molecules. The polynucleotide compositions described herein may include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications can include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). This may also include synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The phrase “genetic profile” is used interchangeably with “genotype information” and refers to part or all of an identified genotype of a subject and may include one or more polymorphisms in one or more genes of interest. A genetic profile may not be limited to specific genes and polymorphisms described herein, and can include any number of other polymorphisms, gene expression levels, polypeptide sequences, or other genetic markers that are associated with a subject or patient.

The term “patient” refers to an individual waiting for or under medical care and treatment, such as a treatment for medical condition. While the disclosed methods are designed for human patients, such methods are applicable to any suitable individual, which includes, but is not limited to, a mammal, such as a mouse, rat, rabbit, hamster, guinea pig, cat, dog, goat, cow, horse, pig, and simian. Human patients include male and female patients of any ethnicity. The term “treating” as used herein is intended to encompass curing as well as ameliorating at least one symptom of a condition or disease.

The nucleic acid codes utilized herein include: A for Adenine, C for Cytosine, G for Guanine, T for Thymine, and U for Uracil.

As used herein, the terms “drug,” “medication,” and “therapeutic compound” or “compound” are used interchangeably and refer to any chemical entity, pharmaceutical, drug, biological, and the like that can be used to treat or prevent a disease, illness, condition, or disorder of bodily function. A drug may comprise both known and potentially therapeutic compounds. A drug may be determined to be therapeutic by screening using the screening known to those having ordinary skill in the art. A “known therapeutic compound” or “medication” refers to a therapeutic compound that has been shown (e.g., through animal trials or prior experience with administration to humans) to be effective in such treatment. Examples of drugs include, but are not limited to peptides, polypeptides, synthetic organic molecules, naturally occurring organic molecules, nucleic acid molecules, and combinations thereof.

The biological basis for an outcome in a specific patient following a treatment with a medication is subject to, inter alia, the patient's genetic predisposition to metabolizing the medication. It has been determined that select polymorphisms of a patient, including single nucleotide permutations, haplotypes and phenotypes may be utilized to generate such genotype information. The genotype information may be utilized to generate prognostic information. The prognostic information may be utilized in determining treatment options for the patient. The prognostic information is then based on the patient's genetic predisposition to treatment based on their drug metabolism for individual drugs or drug combinations. The prognostic information may also be utilized in determining an expected outcome of a treatment of an individual, such as a treatment with the medication.

When a genetic marker such as a polymorphism is used as a basis for determining a treatment for a patient, as described herein, the genetic marker may be measured before or during treatment. The prognostic information obtained may be used by a clinician in assessing any of the following: (a) a probable or likely suitability of an individual to initially receive a drug treatment(s); (b) a probable or likely unsuitability of an individual to initially receive a drug treatment(s); (c) a responsiveness of an individual to a drug treatment; (d) a probable or likely suitability of an individual to continue to receive a drug treatment(s); (e) a probable or likely unsuitability of an individual to continue to receive a drug treatment(s); (f) adjusting dosage of an individual receiving a drug; and (g) predicting likelihood of clinical benefits of an individual receiving a drug. As understood by one of skill in the art, measurement of a genetic marker or polymorphism in a clinical setting can be an indication that this parameter may be used as a basis for initiating, continuing, adjusting and/or ceasing administration of a drug, such as described herein.

Select polymorphisms have been identified that may be utilized for providing prognostic information, according to the principles of the invention. These findings were correlated with various magnitudes of a positive or negative predispositions to drug metabolism risk. Accordingly, assaying the genotype at these markers may be utilized to generate prognostic information that may be utilized to predict the expected outcome of treating the patient with a drug based on the expected predisposition of the patient to drug metabolism risk associated with the drug. Clinicians prescribing medications may utilize the prognostic information to improve therapeutic decisions and to avoid treatment failures.

Many of the known human single nucleotide permutations (SNPs) are catalogued by the National Center for Biotechnology Information (NCBI) in the Reference SNP (i.e., “refSNP”) database maintained by NCBI. The Reference SNP database is a polymorphism database (dbSNP), which includes single nucleotide polymorphisms and related polymorphisms, such as deletions and insertions of one or more nucleotides. The database is a public-domain archive maintained by NCBI for a broad collection of simple genetic polymorphisms and can be accessed at http://www.ncbi.nlm.nih.gov/snp.

A number of patients have experienced health problems associted with the lack of efficacy of certain drugs in specific individuals. Numerous investigations have demonstrated that this phenomenon may be, in part, attributed to the broad variability in individual response profiles and to genetic polymorphisms in candidate genes involved in immunological and inflammatory signaling pathways. Using these polymorphisms to identify patients at risk of irregular drug metabolism would play an important role in modulating drug therapies.

DNA polymorphisms have been identified that may be utilized according to the principles of the invention include SNPs and haplotypes associated with genetic markers in several genes. The genes include the respective genes encoding cytochrome P450 family 2 subfamily C member 8 (CYP2C8), cytochrome P450 family 2 subfamily C member 9 (CYP2C9), cytochrome P450 family 2 subfamily C member 19 (CYP2C19), cytochrome P450 family 3 subfamily A member 4 (CYP3A4), cytochrome P450 family 3 subfamily A member 5 (CYP3A5), cytochrome P450 family 2 subfamily D member 6 (CYP2D6), cytochrome P450 family 1 subfamily A member 2 (CYP1A2), vitamin K epoxide reductase complex subunit 1 (VKORC1), UDP glucuronosyltransferase family 2 member B7 (UGT2B7), cytochrome P450 family 2 subfamily B member 6 (CYP2B6) and cytochrome P450 family 2 subfamily E member 1 (CYP2E1).

For example, a method provided by the invention is a diagnostic method for determining the drug metabolism risk associated with a patient which method is not practised on the patient's body, i.e. is an ex vivo diagnostic method. The method may involve determining patient information which may be obtained by assaying a sample of genetic material associated with the patient. The method does not involve obtaining the sample from the patient's body. The invention also provides uses of the systems and methods, for example of the diagnostic assays, for determining the drug metabolism risk associated with a patient.

Drug Metabolism Algorithm

The drug metabolism tests evaluate a panel of genetic markers to predict good or poor responders to medications. The tests is directed to patients who are taking or candidates for taking medications metabolized by cytochrome P-450 enzymes, UDP-Glucuronosyltransferase-2B7 (UGT2B7), and vitamin K epoxide reductase complex subunit 1 (VKORC1) In order to avoid adverse drug events, physicians may also use this test to assist with prescribing medications at optimal doses. Specific drugs are described below.

For CYP haplotypes, with respect to drug metabolism risk assessment, an exemplary algorithm for determining drug metabolism side effect risk is shown below. Each category is scored separately as shown in the charts below, but all are based on the following scoring system. As would be known by one of ordinary skill in the art, there are four general categories of CYP star alleles (i.e., CYP haplotypes): normal function, reduced function, null function and increased function. The nomenclature is reported by, for example, Robarge et al., “The Star-Allele Nomenclature: Retooling for Translational Genomics” Nature, v. 82, no. 3, September 2007, pp. 244-248, incorporated by reference herein.

A large number of star alleles have been reported for each cytochrome. Among these are normal functioning CYP star alleles, CYP star alleles with some function that is a reduced function, CYP star alleles with null (or non-functional) alleles, and CYP star alleles with increased functionality. These alleles convey a wide range of enzyme activity, from no activity to ultrarapid metabolism of substrates/medications.

Table 1 below describes individual SNPs and haplotypes associated with each gene. Once the haplotypes are identified in each gene, the variants are scored and graded to deduce which activity levels and grades are associated with a patient. The graded variants are then used to identify drug and dosage reccomendfations for a patient. The subtables under table 1 below provides allele and haplotype identification, scoring and grading for CYP2C8, CYP2C9, CYP2C19, CYP3A4, CYP3A5, CYP2D6, CYP1A2, VKORC1, UGT2B7, UGT2B15, CYP2B6 and CYP2E1, respectively.

TABLE 1
A—CYP2C8 alleles, haplotypes, scoring and grading@

| CYP2C8 Haplotype SNPs by Individual DNA Strand | | | | |
|---|---|---|---|---|
| Haplotype Id | CYP2C8 | rs11572103 | rs10509681 | rs1058930 |
| PA165958681 | *1A | T | T | G |
| PA165958682 | *1B | T | T | G |
| PA165958683 | *1C | T | T | G |
| PA165958684 | *2 | A | T | G |
| PA165958685 | *3 | T | C | G |
| PA165958686 | *4 | T | T | C |
| PA165958687 | *5 | T | T | G |
| PA165958688 | *6 | T | T | G |
| PA165958689 | *7 | T | T | G |
| PA165958690 | *8 | T | T | G |
| PA165958691 | *9 | T | T | G |
| PA165958692 | *10 | T | T | G |
| PA165958693 | *11 | T | T | G |
| PA165958694 | *12 | T | T | G |
| PA165958695 | *13 | T | T | G |
| PA165958696 | *14 | T | T | G |

| CYP2C8: Allele Pair ID Scores | | |
|---|---|---|
| NONE = 0 | DECREASED = 0.5 | NORMAL = 1 |
| *5 | *2 | *1A |
| | *3 | |
| | *4 | |

| CYP2C8: Allele Pair ID Grade | |
|---|---|
| C | 1.0 = reduced/reduced |
| C | 1.5 = reduced/functional |
| B | 2 = functional/functional |

Score allele pairs using Table. Score for each allele will be 0 (null function), 0.5 (decreased function), or 1 (normal function).
Sum allele scores to get an “Activity Score” and use this in Table 2 to obtain final grade.
@Unless otherwise indicated, context sequences in FASTA format, are presented by
NCBI within the rs cluster report identified by “rs#” associated with each rs number in
Table 1A above in the NCBI SNP reference database accessible at
http://www.ncbi.nlm.nih.gov/snp, and which is incorporated by reference herein for
each recited SNP rs number in the Table(s) above.
B—CYP2C9 alleles, haplotypes, scoring and grading@

| CYR2C9 | rs1799853 | rs1057910 | rs28371686 | rs9332161 | rs7900194 | rs28371635 | rs72558187 |
|---|---|---|---|---|---|---|---|
| *1 | C | A | C | A | C | C | T |
| *2 | T | A | C | A | G | C | T |
| *3 | C | C | C | A | G | C | T |
| *4 | C | A | C | A | G | C | T |
| *5 | C | A | C | A | G | C | T |
| *6 | C | A | C | [symbol] A | G | C | T |
| *7 | C | A | C | A | G | C | T |
| *8 | C | A | C | A | A | C | T |
| *9 | C | A | C | A | G | C | T |
| *10 | C | A | C | A | G | C | T |
| *11 | C | A | C | A | G | T | T |
| *12 | C | A | C | A | G | C | T |
| *13 | C | A | C | A | G | C | C |
| *14 | C | A | C | A | G | C | T |
| *15 | C | A | C | A | G | C | T |
| *16 | C | A | C | A | G | C | T |
| *17 | C | A | C | A | G | C | T |
| *18 | C | C | C | A | G | C | T |
| *19 | C | A | C | A | G | C | T |
| *20 | C | A | C | A | G | C | T |
| *21 | C | A | C | A | G | C | T |
| *22 | C | A | C | A | G | C | T |
| *23 | C | A | C | A | G | C | T |
| *24 | C or T | A | C | A | G | C | T |
| *25 | C | A | C | A | G | C | T |
| *26 | C | A | C | A | G | C | T |
| *27 | C | A | C | A | G | C | T |
| *28 | C | A | C | A | G | C | T |
| *29 | C | A | C | A | G | C | T |
| *30 | C | A | C | A | G | C | T |
| *31 | C | A | C | A | G | C | T |
| *32 | C | A | C | A | G | C | T |
| *33 | C | A | C | A | G | C | T |
| *34 | C | A | C | A | G | C | T |
| *35 | T | A | C | A | G | C | T |
| *36 | C | A | C | A | G | C | T |
| *37 | C | A | C | A | G | C | T |
| *38 | C | A | C | A | G | C | T |
| *39 | C | A | C | A | G | C | T |
| *40 | C | A | C | A | G | C | T |
| *41 | C | A | C | A | G | C | T |
| *42 | C | A | C | A | G | C | T |
| *43 | C | A | C | A | G | C | T |
| *44 | C | A | C | A | G | C | T |
| *45 | C | A | C | A | G | C | T |
| *46 | C | A | C | A | G | C | T |
| *47 | C | A | C | A | G | C | T |
| *48 | C | A | C | A | G | C | T |
| *49 | C | A | C | A | G | C | T |
| *50 | C | A | C | A | G | C | T |
| *51 | C | A | C | A | G | C | T |
| *52 | C | A | C | A | G | C | T |
| *53 | C | A | C | A | G | C | T |
| *54 | C | A | C | A | G | C | T |
| *55 | C | A | C | A | G | C | T |
| *56 | C | A | C | A | G | C | T |
| *57 | C | A | C | A | G | C | T |
| *58 | C | A | C | A | G | C | T |

| CYP2C9: Activity Scores per allele | | | |
|---|---|---|---|
| NORMAL = 1 | NULL = 0 | INCREASED = 1.5 | DECREASED = 0.5 |
| *1A | *6 | | *3 |
| *1 | *35 in vitro | | *5 |
| | *15 | | *8 |
| | *25 | | *11 |
| | | | *13 |
| | | | *2 |
| | | | *18 |
| | | | *4 |
| | | | *12 |
| | | | *14 |
| | | | *16 |
| | | | *17 |
| | | | *33 |
| | | | *26 |
| | | | *28 |
| | | | *30 |
| | | | *33 |
| | | | *24 |

| CYP2C9: Grading by Activity Scores | |
|---|---|
| D | 0 = null/null |
| D | 0.5 = null/reduced function |
| D | 1 = reduced/reduced OR normal/null |
| C | 1.5 = reduced/normal |
| B | 2 = normal/normal |
| A | >2 = more than 2 normal |

Score allele using Table 1. Score for each allele will be 0 (null function), 0.5 (decreased function), or 1 (normal function).
If allele result is *2 or *35 or *24, score for that allele is “0 or 0.5”
If allele result is *3 or *18, score for that allele is 0.5
Sum allele scores to get an “Activity Score” and use this in Grading Table to obtain final grade.
@Unless otherwise indicated, context sequences in FASTA format, are presented by NCBI
within the rs cluster report identified by “rs#” associated with each rs number in Table 1B above
in the NCBI SNP reference database accessible at http://www.ncbi.nlm.nih.gov/snp, and which is
incorporated by reference herein for each recited SNP rs number in the Table(s) above
C—CYP2C19 alleles, haplotypes, scoring and grading@

| CYP2C19 | rs4244285 | rs4986893 | rs28399504 | rs56337013 | rs72552267 | rs72558186 | rs41291556 | rs12248560 |
|---|---|---|---|---|---|---|---|---|
| *1 | G | G | A | C | G | T | T | C |
| *1A | G | G | A | C | G | T | T | C |
| *1B | G | G | A | C | G | T | T | C |
| *1C | G | G | A | C | G | T | T | C |
| *2 | A | G | A | C | G | T | T | C |
| *2A | A | G | A | C | G | T | T | C |
| *2B | A | G | A | C | G | T | T | C |
| *2C | A | G | A | C | G | T | T | C |
| *2D | A | G | A | C | G | T | T | C |
| *2E | A | G | A | C | G | T | T | C |
| *2F | A | G | A | C | G | T | T | C |
| *2G | A | G | A | C | G | T | T | C |
| *2H | A | G | A | C | G | T | T | C |
| *2J | A | G | A | C | G | T | T | C |
| *3 | G | A | A | C | G | T | T | C |
| *3A | G | A | A | C | G | T | T | C |
| *3B | G | A | A | C | G | T | T | C |
| *3C | G | A | A | C | G | T | T | C |
| *4 | G | G | G | C | G | T | T | C |
| *4A | G | G | G | C | G | T | T | C |
| *4B | G | G | G | C | G | T | T | T |
| *5 | G | G | A | T | G | T | T | C |
| *5A | G | G | A | T | G | T | T | C |
| *5B | G | G | A | T | G | T | T | C |
| *6 | G | G | A | C | A | T | T | C |
| *7 | G | G | A | C | G | A | T | C |
| *8 | G | G | A | C | G | T | C | C |
| *9 | G | G | A | C | G | T | T | C |
| *10 | G | G | A | C | G | T | T | C |
| *11 | G | G | A | C | G | T | T | C |
| *12 | G | G | A | C | G | T | T | C |
| *13 | G | G | A | C | G | T | T | C |
| *14 | G | G | A | C | G | T | T | C |
| *15 | G | G | A | C | G | T | T | C |
| *16 | G | G | A | C | G | T | T | C |
| *17 | G | G | A | C | G | T | T | T |
| *18 | G | G | A | C | G | T | T | C |
| *19 | G | G | A | C | G | T | T | C |
| *22 | G | G | A | C | G | T | T | C |
| *23 | G | G | A | C | G | T | T | C |
| *24 | G | G | A | C | G | T | T | C |
| *25 | G | G | A | C | G | T | T | C |
| *26 | G | G | A | C | G | T | T | C |
| *27 | G | G | A | C | G | T | T | C |
| *28 | G | G | A | C | G | T | T | C |
| *29 | G | G | A | C | G | T | T | C |
| *30 | G | G | A | C | G | T | T | C |
| *31 | G | G | A | C | G | T | T | C |
| *32 | G | G | A | C | G | T | T | C |
| *33 | G | G | A | C | G | T | T | C |
| *34 | G | G | A | C | G | T | T | C |

| CYP2C19: Star Allele Scoring | | | |
|---|---|---|---|
| NULL = 0 | INCREASED = 1.5 | DECREASED = 0.5 | NORMAL = 1 |
| *2A | *17 | | *1 |
| *2B | | | *1A |
| *3A | | | *1B |
| *3B | | | *1C |

*4A
*4B
*5AB
*6
*7
*8
*2
*3
*4

| CYP2C19: Grading by added Activity Score | |
|---|---|
| D | 0 = null/null |
| C | 1 = null/normal |
| C | 1.5 = increased/null |
| B | 2 = normal/normal |
| A | >2 = increased/normal |

Score alleles using Table 1. Score for each allele will be 0 (null function), 0.5 (decreased function), or 1 (normal function).
Sum allele scores to get an “Activity Score” and use this in Table 2 to obtain final grade.
@Unless otherwise indicated, context sequences in FASTA format, are presented by NCBI
within the rs cluster report identified by “rs#” associated with each rs number in Table 1C above
in the NCBI SNP reference database accessible at http://www.ncbi.nlm.nih.gov/snp, and which is
incorporated by reference herein for each recited SNP rs number in the Table(s) above
D—CYP3A4 alleles, haplotypes, scoring and grading@

| CYP3A4 | rs2740574 | rs55785340 | rs4987161 | rs28371759 | rs12721629 | rs35599367 |
|---|---|---|---|---|---|---|
| *1 | T | A | A | A | G | G |
| *1A | T | A | A | A | G | G |
| *1B | C | A | A | A | G | G |
| *1C | T | A | A | A | G | G |
| *1D | T | A | A | A | G | G |
| *1E | T | A | A | A | G | G |
| *1F | T | A | A | A | G | G |
| *1G | T | A | A | A | G | G |
| *1H | T | A | A | A | G | G |
| *1J | T | A | A | A | G | G |
| *1K | T | A | A | A | G | G |
| *1L | T | A | A | A | G | G |
| *1M | T | A | A | A | G | G |
| *1N | T | A | A | A | G | G |
| *1P | T | A | A | A | G | G |
| *1Q | T | A | A | A | G | G |
| *1R | T | A | A | A | G | G |
| *1S | T | A | A | A | G | G |
| *1T | T | A | A | A | G | G |
| *2 | T | G | A | A | G | G |
| *3 | T | A | A | A | G | G |
| *4 | T | A | A | A | G | G |
| *5 | T | A | A | A | G | G |
| *6 | T | A | A | A | G | G |
| *7 | T | A | A | A | G | G |
| *8 | T | A | A | A | G | G |
| *9 | T | A | A | A | G | G |
| *10 | T | A | A | A | G | G |
| *11 | T | A | A | A | G | G |
| *12 | T | A | A | A | A | G |
| *13 | T | A | A | A | G | G |
| *14 | T | A | A | A | G | G |
| *15 | T | A | A | A | G | G |
| *15A | T | A | A | A | G | G |
| *15B | C | A | A | A | G | G |
| *16 | T | A | A | A | G | G |
| *16A | T | A | A | A | G | G |
| *16B | T | A | A | A | G | G |
| *17 | T | A | G | A | G | G |
| *18 | T | A | A | G | G | G |
| *18A | T | A | A | G | G | G |
| *18B | T | A | A | G | G | G |
| *19 | T | A | A | A | G | G |
| *20 | T | A | A | A | G | G |
| *21 | T | A | A | A | G | G |
| *22 | T | A | A | A | G | A |
| *23 | C | A | A | A | G | G |
| *24 | C | A | A | A | G | G |

| CYP3A4: Activity Scores | | | |
|---|---|---|---|
| INCREASED = 1.5 | DECREASED = 0.5 | NORMAL = 1 | NONE = 0 |
| *18 | *1B | *1A | *22 |
| | *8 | *1 | |
| | *11 | *3 | |
| | *12 | *7 | |
| | *13 | *9 | |
| | *16 | *10 | |
| | *17 | *19 | |
| | *2 | | |

| CYP3A4: Grading based on Activity Scores | |
|---|---|
| D | 0 = null/null |
| C | 0.5 = null/reduced |
| C | 1 = reduced/reduced OR normal/null |
| B | 1.5 = reduced/normal |
| B | 2-2.5 = normal/normal or normal/increased |
| A | >2.5 = increased/increased |

Score allele pairs using Table. Score for each allele will be 0 (null function), 0.5 (decreased function), or 1 (normal function).
If allele pairs are “*1B or *15B or *23 or *24”, then report “*1B” and address the result in the disclaimer.
Sum allele scores to get an “Activity Score” and use this in grading Table to obtain final grade.
@Unless otherwise indicated, context sequences in FASTA format, are presented by NCBI
within the rs cluster report identified by “rs#” associated with each rs number in Table 1D above
in the NCBI SNP reference database accessible at http://www.ncbi.nlm.nih.gov/snp, and which is
incorporated by reference herein for each recited SNP rs number in the Table(s) above
E—CYP3A5 alleles, haplotypes, scoring and grading@

| CYP3A5 | TAG(S) | rs776746 | rs41303343 | rs10264272 | rs55817950 | rs28383479 |
|---|---|---|---|---|---|---|
| *1A | | T | del | C | G | C |
| *1B | | T | del | C | G | C |
| *1C | | T | del | C | G | C |
| *1D | | T | del | C | G | C |
| *1E | | T | del | C | G | C |
| *2 | rs28365083 | T | del | C | G | C |
| *3A | rs776746 | C | del | C | G | C |
| *3B | rs776746 | C | del | C | G | C |
| *3C | rs776746 | C | del | C | G | C |
| *3D | rs776746 | C | del | C | G | C |
| *3E | rs776746 | C | del | C | G | C |
| *3F | rs776746 | C | del | C | G | C |
| *3G | rs776746 | C | del | C | G | C |
| *3H | rs776746 | C | del | C | G | C |
| *3I | rs776746 | C | del | C | G | C |
| *3J | rs776746 | C | del | C | G | C |
| *3K | rs776746 | C | del | C | G | C |
| *3L | rs776746 | C | del | C | G | C |
| *4 | rs56411402 | T | del | C | G | C |
| *5 | | T | del | C | G | C |
| *6 | rs10264272 | T | del | T | G | C |
| *7 | rs41303343 | T | A | C | G | C |
| *8 | rs55817950 | T | del | C | A | C |
| *9 | rs28383479 | T/C | del | C | G | T |

CYP3A5: Star Allele Activity Scoring

| INCREASED | DECREASED = 0.5 | NORMAL = 1 | NONE = 0 | CYP3A5: Grading based on Activity Score | |
|---|---|---|---|---|---|
| none | *8 | *1A | *7 | D | 0 = null/null |
| | *9 | *1 | *3 | D | 0.5 = null/reduced function |
| | | *2 | *6 | C | 1 = reduced/reduced OR normal/null |
| | | *4 | | B | 1.5 = reduced/normal |
| | | *5 | | B | 2 = normal/normal |

Score alleles using Table. Score for each allele will be 0 (null function), 0.5 (decreased function), or 1 normal function).
Sum allele scores to get an “Activity Score” and use this to obtain final grade.
@Unless otherwise indicated, context sequences in FASTA format, are presented by NCBI
within the rs cluster report identified by “rs#” associated with each rs number in Table lE above
in the NCBI SNP reference database accessible at http://www.ncbi.nlm.nih.gov/snp, and which is
incorporated by reference herein for each recited SNP rs number in the Table(s) above
F—CYP2D6 alleles, haplotypes, scoring and grading@

| CYP2D6 | | rs1080985 | rs35742686 | rs3892097 | rs5030655 | rs5030867 | rs5030865 | rs1065852 | | |
|---|---|---|---|---|---|---|---|---|---|---|
| *1 | | G | T | C | A | T | C | G | | |
| *71 | | C | T | C | A | T | C | G | | |
| *15 | | G | T | C | A | T | C | G | | |
| Report *39 (default). | | G | T | C | A | T | C | G | | |
| *70 | | G | T | C | A | T | C | G | | |
| *7 | | G | T | C | A | G | C | G | | |
| *34 | | G | T | C | A | T | C | G | | |
| *63 | | C | T | C | A | T | C | G | | |
| Report *2 (default). | | G | T | C | A | T | C | G | | |
| *20 | | G | T | C | A | T | C | G | | |
| *14 | | G | T | C | A | T | T | G | | |
| *11 | | G | T | C | A | T | C | G | | |
| Report “*2A or *21”. | | C | T | C | A | T | C | G | | |
| *29 | | G | T | C | A | T | C | G | | |
| *91 | | G | T | C | A | T | C | G | | |
| *41 | | G | T | C | A | T | C | G | | |
| *9 | | G | T | C | A | T | C | G | | |
| Report *3 (default). | | G | dell | C | A | T | C | G | | |
| *4M | | G | T | T | A | T | C | G | | |
| *6 | | G | T | C | delA | T | C | G | | |
| *6C | | G | T | C | delA | T | C | G | | |
| Report *17 (default). | | G | T | C | A | T | C | G | | |
| *12 | | G | T | C | A | T | C | G | | |
| *68A | | G | T | C | A | T | C | A | | |
| Report “*10 or *36”. | | G | T | C | A | T | C | A | | |
| *65 | | G | T | C | A | T | C | A | | |
| *14A | | G | T | C | A | T | T | A | | |
| *69 | | G | T | C | A | T | C | A | | |
| Report *4 (default). | | G | T | T | A | T | C | A | | |
| Report *4 (default). | | G | T | T | A | T | C | A | | |
| *4K | | G | T | T | A | T | C | A | | |
| *64 | | G | T | C | A | T | C | A | | |
| Report *35 (default). | | C | T | C | A | T | C | G | | |
| rs72549385 | rs28371706 | rs59421388 | rs769258 | rs16947 | rs1135840 | rs28371725 | rs5030656 | rs201377835 | rs5030862 | rs7254935 |
| del | G | C | C | G | C | C | CTT | C | C | del |
| del | G | C | C | G | C | C | CTT | C | C | del |
| insA | G | C | C | G | C | C | CTT | C | C | del |
| del | G | C | C | G | G | C | CTT | C | C | del |
| del | G | T | C | G | G | C | CTT | C | C | del |
| del | G | C | C | G | C | C | CTT | C | C | del |
| del | G | C | C | A | C | C | CTT | C | C | del |
| del | G | C | C | A | C | C | CTT | C | C | del |
| del | G | C | C | A | G | C | CTT | C | C | del |
| del | G | C | C | A | G | C | CTT | C | C | insC |
| del | G | C | C | A | G | C | CTT | C | C | del |
| del | G | C | C | A | G | C | CTT | G | C | del |
| del | G | C | C | A | G | C | CTT | C | C | del |
| del | G | T | C | A | G | C | CTT | C | C | del |
| del | G | C | C | A | C | T | CTT | C | C | del |
| del | G | C | C | A | G | T | CTT | C | C | del |
| del | G | C | C | G | C | C | delCTT | C | C | del |
| del | G | C | C | G | C | C | CTT | C | C | del |
| del | G | C | C | G | C | C | CTT | C | C | del |
| del | G | C | C | G | C | C | CTT | C | C | del |
| del | G | C | C | G | G | C | CTT | C | C | del |
| del | A | C | C | A | G | C | CTT | C | C | del |
| del | G | C | C | A | G | C | CTT | C | T | del |
| del | G | C | C | G | C | C | CTT | C | C | del |
| del | G | C | C | G | G | C | CTT | C | C | del |
| del | G | C | C | A | G | C | CTT | C | C | del |
| del | G | C | C | A | G | C | CTT | C | C | del |
| del | G | C | C | A | G | T | CTT | C | C | del |
| del | G | C | C | G | C | C | CTT | C | C | del |
| del | G | C | C | G | G | C | CTT | C | C | del |
| del | G | C | C | A | G | C | CTT | C | C | del |
| del | A | C | C | G | G | C | CTT | C | C | del |
| del | G | C | T | A | G | C | CTT | C | C | del |
| CYP2D6: Star Allele Scoring | | | | | | | | | | |
| increased = 1.5 | reduce = 0.5 | null = 0 | normal = 1 | | | | | | | |
| *1XN | *9 | *3 | *1 | | | | | | | |
| *2XN | *10 | *4A-D | *2A | | | | | | | |
| *35X2 | *10X2 | *4K | *33 | | | | | | | |
| | *17 | *4X2 | *35 | | | | | | | |
| | *29 | *5 | *27 | | | | | | | |
| | *41 | *6 | *33 | | | | | | | |
| | *69 | *7 | *35 | | | | | | | |
| | *91 | *8 | *45 | | | | | | | |
| | *49 | *11 | *46 | | | | | | | |
| | *50 | *12 | *39 | | | | | | | |
| | *54 | *13 | *48 | | | | | | | |
| | *55 | *14 | *53 | | | | | | | |
| | *59 | *15 | *2 | | | | | | | |
| | *69 | *18 | *71 | | | | | | | |
| | *72 | *19 | *70 | | | | | | | |
| | *14B | *20 | *34 | | | | | | | |
| | | *21A-B | *63 | | | | | | | |
| | | *31 | *65 | | | | | | | |
| | | *38 | *64 | | | | | | | |
| | | *40 | | | | | | | | |
| | | *42 | | | | | | | | |
| | | *44 | | | | | | | | |
| | | *4 | | | | | | | | |
| | | *16 | | | | | | | | |
| | | *36 | | | | | | | | |
| | | *47 | | | | | | | | |
| | | *51 | | | | | | | | |
| | | *56 | | | | | | | | |
| | | *57 | | | | | | | | |

```
*62
  *4M
  *6C
*14A
  *3A
  *6A
  *6B
*68A
```

| | CYP2D6: Grading | | |
|---|---|---|---|
| | Activity | | |
| Grade | score | Functional Allele diplotype | [symbol] |
| D | 0 | null/null | [symbol] |
| D | 0.5 | null/reduced | [symbol] |
| D | 1 | reduced/reduced OR normal/nu [symbol] | [symbol] |
| C | 1.5 | normal/reduced function | [symbol] |
| B | 2 | normal/normal | [symbol] |
| A | >2 | duplicates of functional alleles | [symbol] |

Score each allele using Table 1. Score for each allele will be either 0 (null function), 0.5 (decreased function), or 1 (normal function).
Sum each allele score to get an “activity score”. Use Activity Score in Table 2 to determine grade.
If your allele diplotype ends up being “*4/*39 or *36/*4M or *10/*4M or *4/*1”, then report “Unknown allele set”. This will be a “possible decreased metabolizer”
@Unless otherwise indicated, context sequences in FASTA format, are presented by NCBI
within the rs cluster report identified by “rs#” associated with each rs number in Table 1F above
in the NCBI SNP reference database accessible at http://www.ncbi.nlm.nih.gov/snp, and which is
incorporated by reference herein for each recited SNP rs number in the Table(s) above
G—CYP1A2 alleles, haplotypes, scoring and grading@

| CYP1A2 | rs762551 | rs2472304 | rs56107638 | rs28399424 | rs2470890 |
|---|---|---|---|---|---|
| *1A | C | G | G | C | T |
| *1B | C | G | G | C | C |
| *1C | C | G | G | C | T |
| *1D | C | G | G | C | T |
| *1E | C | G | G | C | T |
| *1F | A | G | G | C | T |
| *1G | C | G | G | C | C |
| *1H | C | G | G | C | C |
| *1J | A | G | G | C | T |
| *1K | A | G | G | C | T |
| *1L | A | G | G | C | C |
| *1M | A | A | G | C | T |
| *1N | A | G | G | C | C |
| *1P | A | G | G | C | C |
| *1O | A | A | G | C | T |
| *1R | A | G | G | C | C |
| *1S | C | G | G | C | C |
| *1T | C | G | G | C | C |
| *1U | C | G | G | C | C |
| *1V | A | G | G | C | T |
| *1W | A | G | G | C | T |
| *2 | C | G | G | C | T |
| *3 | C | G | G | C | C |
| *4 | C | G | G | C | T |
| *5 | C | G | G | C | T |
| *6 | C | G | G | T | T |
| *7 | C | G | A | C | T |
| *8 | C | G | G | C | C |
| *9 | C | G | G | C | T |
| *10 | C | G | G | C | T |
| *11 | C | G | G | C | T |
| *12 | C | G | G | C | T |
| *13 | C | G | G | C | T |
| *14 | C | G | G | C | T |
| *15 | C | G | G | C | C |
| *16 | C | G | G | C | C |
| *17 | A | A | G | C | C |
| *18 | C | G | G | C | C |
| *19 | C | G | G | C | C |
| *20 | C | G | G | C | C |
| *21 | A | G | G | C | C |

| CYP1A2: Star Allele Scoring | | | |
|---|---|---|---|
| NULL = 0 | INCREASED = 1.5 | DECREASED = 0.5 | NORMAL = 1 |
| *6 | *1F | *1C | *1A |
| | | *1K | *1M |
| | | *3 | *1Q |
| | | *4 | *17 |
| | | *7 | *1L |
| | | *8 | *1N |
| | | *11 | *1P |
| | | *15 | *1R |
| | | *16 | *21 |
| | | | *1B |

| CYP2D6: Grading based on Activity Score | |
|---|---|
| D | 0 = null/null |
| D | 0.5 = null/reduced function |
| C | 1 = reduced/reduced OR normal/null |
| B | 1.5 = reduced/normal |
| B | 2 = normal/normal |
| A | 2.5 = increased/normal |
| A | 3 = increased/increased |

Score allele pairs using Table. Score for each allele will be 0 (null function), 0.5 (decreased function), or 1 (normal function).
Sum allele scores to get an “Activity Score” and use this to obtain final grade.
@Unless otherwise indicated, context sequences in FASTA format, are presented by NCBI
within the rs cluster report identified by “rs#” associated with each rs number in Table 1G above
in the NCBI SNP reference database accessible at http://www.ncbi.nlm.nih.gov/snp, and which is
incorporated by reference herein for each recited SNP rs number in the Table(s) above
1H—VKORC1 alleles, haplotypes, scoring and grading@

| VKORC1 | rs9923231 | rs17708472 | rs9934438 | rs2359612 | rs7294 |
|---|---|---|---|---|---|
| *1 | C | G | G | G | C |
| *2 | T | G | A | A | C |
| *3 | C | G | G | G | T |
| *4 | C | A | G | G | C |
| | | activity score | grade | | |
| VKORC1 | *1/*1 | 2 | b | | |
| Allele Set | *1/*2 | 1.5 | c | | |
| | *1/*3 | 2.5 | b | | |
| | *1/*4 | 2.5 | b | | |
| | *2/*2 | 1 | d | | |
| | *2/*3 | 2 | b | | |
| | *2/*4 | 2 | b | | |
| | *3/*3 | 3 | a | | |
| | *3/*4 | 3 | a | | |
| | *4/*4 | 3 | a | | |

@Unless otherwise indicated, context sequences in FASTA format, are presented by NCBI
within the rs cluster report identified by “rs#” associated with each rs number in Table 1H above
in the NCBI SNP reference database accessible at http://www.ncbi.nlm.nih.gov/snp, and which is
incorporated by reference herein for each recited SNP rs number in the Table(s) above
I—UGT2B7 and UGT2B15 alleles, haplotypes, scoring and grading@

| SNP: | | Genotype | Report |
|---|---|---|---|
| UGT2B7 | | | |
| rs6600880 | (−1759T > A) | T/T | Normal |
| | | T/A | Increased severity of withdrawal symptoms in methadone maintenance |
| | | A/A | Increased severity of withdrawal symptoms in methadone maintenance |
| rs6600879 | (−1852C > G) | C/C | Normal |
| | | C/G | Increased severity of withdrawal symptoms in methadone maintenance |
| | | G/G | Increased severity of withdrawal symptoms in methadone maintenance |
| rs7668282 | (−79G > A, | TT | Decreased clearance of medications. |
| | −125T > C)*1b,*2g | CT | Normal clearance of medications. |
| | | CC | Normal clearance of medications. |
| rs7668258 | (−161C > T) | CC | Normal clearance of medications. |
| | | CT | Decreased clearance of medications. |
| | | TT | Decreased clearance of medications. |
| UGT2B15 | | | |
| rs1902023 | *2 and *5, Y85D, | CC (DD) | Normal |

| | | |
|---|---|---|
| 291G/T | CA | Moderate reduction in clearance is possible. Consider dose decrease. |
| | AA (YY) | Reduced clearance. Consider dose decrease. |

J—CYP2B6 alleles, haplotypes, scoring and grading@

| CYP2B6 | | | |
|---|---|---|---|
| SNP: | Allele ID: | Genotype | PHENOTYPE |
| rs3745274 | B6 | G/G | Normal metabolism |
| | | G/T | Normal metabolism |
| | | T/T | Decreased metabolism |

@Unless otherwise indicated, context sequences in FASTA format, are presented by NCBI
within the rs cluster report identified by “rs#” associated with each rs number in Table 1I and IJ
above in the NCBI SNP reference database accessible at http://www.ncbi.nlm.nih.gov/snp, and
which is incorporated by reference herein for each recited SNP rs number in the Table(s) above.
1K—CYP2E1 alleles, haplotypes, scoring and grading@

| CYP2E1 | Tag(s) | rs2070676 | rs72559710 | rs55897648 | rs6413419 | rs3813867 | rs2031920 | 7632T > A | -333T > A | -71G > T | -352A > G |
|---|---|---|---|---|---|---|---|---|---|---|---|
| *1 | | C | G | G | G | G | C | T | T | G | A |
| *1A | | C | G | G | G | G | C | T | T | G | A |
| *1B | | G | | | | | | | | | |
| *2 | | | A | | | | | | | | |
| *3 | | | | A | | | | | | | |
| *4 | | | | | A | | | | | | |
| *5A | | | | | | C | T | A | | | |
| *5B | | | | | | C | T | | | | |
| *6 | | | | | | | | A | | | |
| *7 | | | | | | | | | A | | |
| *7A | | | | | | | | | A | | |
| *7B | | | | | | | | | A | T | |
| *7C | | | | | | | | | A | | G |

| CYP2E1: Allele Scores | | | |
|---|---|---|---|
| INCREASED | DECREASE = 0.5 | NORMAL = 1 | NONE = 0 |
| | *2 | *1A | |
| | | *1 | |
| | | *3 | |
| | | *4 | |

| CYP2E1: Grading based on Activity Score | |
|---|---|
| D | 0 = null/null |
| D | 0.5 = null/reduced function |
| C | 1 = reduced/reduced OR normal/null |
| B | 1.5 = reduced/normal |
| B | 2 = normal/normal |
| A | 2.5 = increased/normal |
| A | 3 = increased/increased |

@Unless otherwise indicated, context sequences in FASTA format, are presented by NCBI
within the rs cluster report identified by “rs#” associated with each rs number in Table 1K above
in the NCBI SNP reference database accessible at http://www.ncbi.nlm.nih.gov/snp, and which is
incorporated by reference herein for each recited SNP rs number in the Table(s) above [symbol]
indicates data missing or illegible when filed

Once the grades are calculated for each cytochrome, the next step is use these results to make a dosing recommendation for each medication. If a medication is only metabolized by 1 cytochrome, this is fairly straightforward. For the most part, a decreased metabolism result will report that the patient is at risk of an adverse drug event for active medications, or at risk of experiencing insufficient relief from their medication for prodrugs. However, if a medication is metabolized by multiple CYP pathways, the recommendation takes into account all the (clinically-significant) pathways. This is determined using a logical approach for each medication and/or type of medication to provide drug metabolism recommendations.

Table 2 below provides drug metabolism recommendations for opioids, non-steroidal anti-inflammatory drugs, benzodiazepines, psychiatric medications, cardiology medications and warfarin.

TABLE 2
A Opioid drug recommendations based on CYP grade

| | | A | B | C | D | |
|---|---|---|---|---|---|---|
| Buprenorphine | CYP3A4 | This patient may | This patient is | This patient is | This patient may | |
| (Butrans) | | require higher doses | predicted to be a | predicted to be a | require lower doses | |
| | | of buprenorphine | normal metabolizer | normal metabolizer | of buprenorphine | |
| | | due to accelerated | of buprenorphine. | of buprenorphine. | due to a decreased | |
| | | metabolism of the | Prescribe at | Prescribe at | metabolism of | |
| | | medication. H * | standard label | standard label | the medication. L * | |
| | | | recommendations. | recommendations. | | |
| | | A | B | C | D | C/D |
| Codeine | CYP2D6 | This patient is at | This patient is | This patient may | This patient may | This patient may |
| | | increased risk of | predicted to be a | experience insufficient | experience insufficient | experience insufficient |
| | | experiencing an | normal metabolizer | pain relief due to | pain relief due to | pain relief due to |
| | | adverse drug event | of codeine. Prescribe | decreased metabolism | decreased metabolism | decreased metabolism to |
| | | with codeine due to | at standard label | to morphine. Monitor | to morphine. Consider | morphine. Monitor the |
| | | increased | recommendations. | the patient's analgesic | prescribing an | patient's analgesic |
| | | metabolism to | F *** | response and consider | alternative medication | response and consider |
| | | morphine. Consider | | prescribing an increased | not metabolized | prescribing an increased |
| | | alternative | | dose if necessary or | by CYP2D6. | dose if necessary or |
| | | medication not | | select alternative | G, Pd, D, F *** | select alternative |
| | | metabolized by | | medication not | | medication not |
| | | CYP2D6. | | metabolized by CYP2D6. | | metabolized by CYP2D6. |
| | | G, Pi, S, D, F *** | | G, Pd, H, D, F *** | | G, Pd, H, D, F *** |

| CYP3A4/5 | | | | | |
|---|---|---|---|---|---|
| combined | | | | | |
| grade | 3A4 B | 3A4 C | 3A4 D | 3A4 NA | 3A4 “U” |
| 3A5 B | A | B | C | NA | A |
| 3A5 C | B | B | D | NA | B |
| 3A5 D | B | C | D | NA | B |
| 3A5 NA or U | B | C | D | NA | B |

| | | A | B | C | D |
|---|---|---|---|---|---|
| Fentanyl | CYP3A4/5 | This patient may | This patient is | This patient may | This patient may |
| (Duragesic) | | experience insufficient | predicted to be a | experience a longer | experience a longer |
| | | pain relief with average | normal metabolizer | duration of analgesia | duration of analgesia with |
| | | doses of Fentanyl. Monitor | of Fentanyl. | with a standard dose | of a standard dose of |
| | | the patient's analgesic | Prescribe at | of Fentanyl. Monitor | Fentanyl. Be alert for |
| | | response and consider | standard label | the patient's analgesic | adverse drug events and |
| | | administering an | recommendations. | response and be alert | consider lowering the dose |
| | | increased dose or | | for adverse drug | of the medication. L** |
| | | changing the dosing | | events. L* | |
| | | frequency if necessary. H | | | |
| | | ** | | | |

CYP3A4 for Hydrocodone

| A | B or NA | C | D |
|---|---|---|---|
| This patient may be at risk | This patient is at risk of | This patient is at risk of | This patient is at risk of |
| of experiencing an adverse | experiencing an adverse | experiencing an adverse | experiencing an adverse |
| drug event with | drug event with | drug event with | drug event with |
| Hydrocodone due to | Hydrocodone due to | Hydrocodone due to | Hydrocodone due to |
| increased metabolism to | increased metabolism to | increased metabolism to | increased metabolism to |
| hydromorphone. Consider a | hydromorphone. Consider a | hydromorphone. Consider a | hydromorphone. Consider a |
| lower starting dose and | lower starting dose or | lower starting dose or | lower starting dose or |
| monitor closely for side | select alternative | select alternative | select alternative |
| effects. If pain relief is | medication (not oxycodone | medication (not oxycodone | medication (not oxycodone |
| insufficient and patient is | or codeine). Pi, S, L, D ** | or codeine). Pi, S, L, D ** | or codeine). Pi, S, L, D ** |
| tolerating the medication | | | |
| well, consider higher | | | |
| maintenance dose. Pi, C, S * | | | |

| | CYP2C19, | | | | |
|---|---|---|---|---|---|
| Methadone | CYP3A4, | | ABCB1 CATEGORY (Frequency) | | |
| (Dolophine) | ABCB1 | | high | avg | low |
| | CYP | high | 6. This patient is predicted | 9. This patient is predicted | 3. This patient is predicted |
| | CATEGORY | | to exhibit lower trough | to exhibit lower peak (post- | to exhibit higher trough |
| | (Quantity) | | (pre-dose) and lower peak | dose) plasma | (pre-dose) and lower peak |
| | [symbol] | | (post-dose) plasma | concentrations of | (post-dose) plasma |
| | based on | | concentrations of | methadone due to genetic | concentrations of |
| | table | | methadone due to genetic | variants in drug | methadone due to genetic |
| | below | | variants in efflux pumps | metabolizing enzymes. | variants in efflux pumps |
| | | | and drug metabolizing | Consider prescribing a | and drug metabolizing |
| | | | enzymes. Consider a | higher dose for methadone- | enzymes. Consider a higher |
| | | | higher dose and frequency | maintenance therapy. | dose and lower frequency |
| | | | for methadone- | | for methadone- |
| | | | maintenance therapy. | | maintenance therapy. |
| | | avg | 5. This patient is predicted | 8. This patient is predicted | 2. This patient is predicted |
| | | | to exhibit lower trough | to require an average | to exhibit higher trough |
| | | | (pre-dose) plasma | methadone maintenance | (pre-dose) plasma |
| | | | concentrations of | treatment dose. | concentrations of |
| | | | methadone due to genetic | | methadone due to genetic |
| | | | variants in p-glycoprotein | | variants in p-glycoprotein |
| | | | efflux pumps. Consider | | efflux pumps. Consider a |
| | | | increasing the frequency | | lower frequency for |
| | | | (i.e. splitting the dose) for | | methadone-maintenance |
| | | | methadone-maintenance | | therapy. |
| | | | therapy. | | |
| | | low | 4. This patient is predicted | 7. This patient is predicted | 1. This patient is predicted |
| | | | to exhibit lower trough | to exhibit higher peak | to exhibit higher trough |
| | | | (pre-dose) and higher peak | (post-dose) plasma | (pre-dose) and higher peak |
| | | | (post-dose) plasma | concentrations of | (post-dose) plasma |
| | | | concentrations of | methadone due to genetic | concentrations of |
| | | | methadone due to genetic | variants in drug | methadone due to genetic |
| | | | variants in efflux pumps | metabolizing enzymes. | variants in efflux pumps |
| | | | and drug metabolizing | Consider a lower dose for | and drug metabolizing |
| | | | enzymes. Consider a lower | methadone-maintenance | enzymes. Consider a lower, |
| | | | dose and higher frequency | therapy. | less frequent dose for |
| | | | for methadone- | | methadone-maintenance |
| | | | maintenance therapy. | | therapy. |

| CYP Category is | | CYP3A4 Grade | | | |
|---|---|---|---|---|---|
| calculated as follows: | | A | B | C | D |
| CYP2C1 | A | high | high | avg | avg |
| 9 Grade | B | avg | avg | avg | low |
| | C | avg | avg | low | low |
| | D | low | low | low | low |

| | |
|---|---|
| Oxymorphone (Opana) | This patient is not predicted to have abnormal metabolism of oxymorphone. Prescribe at standard label recommendations. |
| Tapentadol (Nucynta) | This patient is not predicted to have abnormal metabolism of Tapentadol. Prescribe at standard label recommendations. |

CYP3A4 cross with CYP2D6 for Tramadol

| | B or NA | C | |
|---|---|---|---|
| This patient is at risk of | This patient is at risk of | This patient is at risk of | This patient is at risk of |
| experiencing an adverse | experiencing an adverse | experiencing an adverse | experiencing an adverse |
| drug event (ADE) with | drug event (ADE) with | drug event (ADE) with | drug event (ADE) with |
| Tramadol. According to | Tramadol. According to | Tramadol. According to | Tramadol. According to |
| published guidelines, | published guidelines, | published guidelines, | published guidelines, |
| consider a 30% | consider a 30% | consider a 30% | consider a 30% |
| decreased dose and be | decreased dose and be | decreased dose and be | decreased dose and be |
| alert for ADEs, or use an | alert for ADEs, or use an | alert for ADEs, or use an | alert for ADEs, or use an |
| alternative to tramadol | alternative to tramadol | alternative to tramadol | alternative to tramadol |
| that is not oxycodone or | that is not oxycodone or | that is not oxycodone or | that is not oxycodone or |
| codeine. G, Pi, L, D, S, F | codeine. G, Pi, L, D, S, F | codeine. G, Pi, L, D, S, F | codeine. G, Pi, L, D, S, F |
| *** | *** | *** | *** |
| This patient is predicted | This patient is predicted | This patient is predicted | This patient may experience |
| to be a normal | to be a normal | to be a normal | reduced clearance of Tramadol. |
| metabolizer of | metabolizer of | metabolizer of | Consider lowering the dose of |
| Tramadol. Prescribe at | Tramadol. Prescribe at | Tramadol. Prescribe at | the medication. L * |
| standard label | standard label | standard label | |
| recommendations. *** | recommendations. *** | recommendations. *** | |
| This patient is predicted | This patient may experience | This patient may experience | This patient may experience |
| to be a normal | reduced clearance of Tramadol. | reduced clearance of Tramadol. | reduced clearance of Tramadol. |
| metabolizer of | Consider lowering the dose of | Consider lowering the dose of | Consider lowering the dose of |
| Tramadol. Prescribe at | the medication. L * | the medication. L * | the medication. L * |
| standard label | | | |
| recommendations. *** | | | |
| This patient has an | This patient is a possible | This patient is a possible | This patient is at risk of |
| unresolved CYP2D6 | decreased metabolizer of | decreased metabolizer of | experiencing an adverse drug |
| phenotype that may be | tramadol into its active | tramadol into its active | event with this medication due |
| normal or may involve | metabolite. Initiate | metabolite. Initiate | to decreased clearance, and |
| decreased enzymatic | recommended starting dose and | recommended starting dose and | possibly may not receive |
| activity. Pain relief may | monitor closely. | monitor closely. | adequate analgesia due to |
| be insufficient with | | | decreased metabolism of |
| tramadol due to | | | Tramadol into its more active |
| decreased metabolism | | | form. Consider alternative |
| into its more active | | | medication (not oxycodone or |
| metabolite. Initiate | | | codeine). G, Pd, S, D, F *** |
| recommended starting | | | |
| dose and monitor | | | |
| closely. | | | |

B NSAID drug recommendations based on CYP grade

| | Metabolizing | CYP Genetic Report | | |
|---|---|---|---|---|
| | enzyme | B | C | D, C/D |
| Celecoxib | CYP2C9 | This patient is predicted to | This patient is at risk of | This patient is at risk of experiencing |
| (Celebrex) | | metabolize this medication | experiencing an adverse drug | an adverse drug event with this |
| | | normally. Prescribe with | event with this medication | medication due to decreased |
| | | standard precautions. | due to decreased metabolism. | metabolism. Consider a 50% |
| | | | Consider a reduction of | reduction of recommended starting |
| | | | recommended starting dose | dose and titrate to response. L,S,F ** |
| | | | and/or reducing frequency | |
| | | | of use. ** | |
| Diclofenac | CYP2C8 | This patient is predicted to | This patient is at risk of | This patient is at risk of experiencing |
| (Voltaren) | | metabolize this medication | experiencing an adverse drug | an adverse drug event with this |
| | | normally. Prescribe with | event with this medication | medication due to decreased |
| | | standard precautions. | due to decreased metabolism. | metabolism. Consider a reduction of |
| | | | Consider a reduction of | recommended starting dose and/or |
| | | | recommended starting dose | reducing frequency of use. ** |
| | | | and/or reducing frequency | |
| | | | of use. ** | |
| Etodolac | CYP2C9 | This patient is predicted to | This patient is at risk of | This patient is at risk of experiencing |
| (Lodine) | | metabolize this medication | experiencing an adverse | an adverse drug event with this |
| | | normally. Prescribe with | drug event with this | medication due to decreased |
| | | standard precautions. | medication due to decreased | metabolism. Consider a reduction of |
| | | | metabolism. Consider a | recommended starting dose and/or |
| | | | reduction of recommended | reducing frequency of use. * |
| | | | starting dose and/or | |
| | | | reducing frequency of use. * | |
| Meloxicam | CYP2C9 | This patient is predicted to | This patient is at risk of | This patient is at risk of experiencing |
| (Mobic) | | metabolize this medication | experiencing an adverse | an adverse drug event with this |
| | | normally. Prescribe with | drug event with this medication | medication due to decreased |
| | | standard precautions. | due to decreased metabolism. | metabolism. Consider a reduction of |
| | | | Consider a reduction of | recommended starting dose and/or |
| | | | recommended starting dose | reducing frequency of use. * |
| | | | and/or reducing frequency | |
| | | | of use. * | |
| | CYP2C9 | | | |
| | (metabolism), | | | |
| | CYP1A2 | | | |
| | (bioactivation) | CYP1A2: A, A/B | CYP1A2: B | CYP1A2: C |
| Nabumetone | CYP2C9: B | This patient is at increased risk | This patient is predicted to | This patient may experience treatment |
| (Relafen) | | of experiencing an adverse drug | metabolize this medication | failure due to decreased bioactivation |
| | | event due to accelerated | normally. Prescribe with | of this medication. Consider higher |
| | | bioactivation of this | standard precautions. | dose if indicated, or select an |
| | | medication. Consider a lower | | alternative medication. Pd, H, D |
| | | dose if indicated. Pi, L | | |
| | CYP2C9: C, D, | This patient is at increased risk | This patient is at risk of | This patient has a complex genotype |
| | C/D | of experiencing an adverse drug | experiencing an adverse | that may be associated with abnormal |
| | | event due to abnormal drug | drug event with this | drug metabolism. Consider an |
| | | metabolism. Consider lower | medication due to decreased | alternative medication. Pd, D |
| | | dose if indicated, or select an | metabolism. Consider a | |
| | | alternative medication. | reduction of recommended | |
| | | Pi, L, S, D | starting dose and | |
| | | | titrate to response. L, S | |

| | | B | C, D, C/D |
|---|---|---|---|
| Flurbiprofen | CYP2C9 | This patient is predicted to | This patient is at risk of experiencing |
| (Ansaid) | | metabolize this medication | an adverse drug event with this |
| | | normally. Prescribe with | medication due to decreased |
| | | standard precautions. | metabolism. Consider a reduction of |
| | | | recommended starting dose and |
| | | | titrate to response. ** |
| Indomethacin | CYP2C9 | This patient is predicted to | This patient is at risk of experiencing |
| (Indocin) | | metabolize this medication | an adverse drug event with this |
| | | normally. Prescribe with | medication due to decreased |
| | | standard precautions. | metabolism. Consider a reduction of |
| | | | recommended starting dose and/or |
| | | | reducing frequency of use. ** |
| Ketoprofen | CYP2C9 | This patient is predicted to | This patient is at risk of experiencing |
| (Actron) | | metabolize this medication | an adverse drug event with this |
| | | normally. Prescribe with | medication due to decreased |
| | | standard precautions. | metabolism. Consider a reduction of |
| | | | recommended starting dose and/or |
| | | | reducing frequency of use. * |
| Ketorolac | CYP2C9 | This patient is predicted to | This patient is at risk of experiencing |
| (Toradol) | | metabolize this medication | an adverse drug event with this |
| | | normally. Prescribe with | medication due to decreased |
| | | standard precautions. | metabolism. Consider a reduction of |
| | | | recommended starting dose and/or |
| | | | reducing frequency of use. * |
| Meclofenamate | CYP2C9 | This patient is predicted to | This patient is at risk of experiencing |
| (Meclomen) | | metabolize this medication | an adverse drug event with this |
| | | normally. Prescribe with | medication due to decreased |
| | | standard precautions. | metabolism. Consider a reduction of |
| | | | recommended starting dose and/or |
| | | | reducing frequency of use. * |
| Mefenamic acid | CYP2C9 | This patient is predicted to | This patient is at risk of experiencing |
| (Ponstel) | | metabolize this medication | an adverse drug event with this |
| | | normally. Prescribe with | medication due to decreased |
| | | standard precautions. | metabolism. Consider a reduction of |
| | | | recommended starting dose and/or |
| | | | reducing frequency of use. * |
| Oxaprozin | CYP2C9 | This patient is predicted to | This patient is at risk of experiencing |
| (Daypro) | | metabolize this medication | an adverse drug event with this |
| | | normally. Prescribe with | medication due to decreased |
| | | standard precautions. | metabolism. Consider a reduction of |
| | | | recommended starting dose and/or |
| | | | reducing frequency of use. ** |
| Piroxicam | CYP2C9 | This patient is predicted to | This patient is at risk of experiencing |
| (Feldene) | | metabolize this medication | an adverse drug event with this |
| | | normally. Prescribe with | medication due to decreased |
| | | standard precautions. | metabolism. Consider a reduction of |
| | | | recommended starting dose and/or |
| | | | reducing frequency of use. ** |

| | | A | B | C, D |
|---|---|---|---|---|
| Tolmetin | CYP3A4 | This patient may experience treatment | This patient is predicted to | This patient is at risk of experiencing |
| sodium | | failure due to increased metabolism | metabolize this medication | an adverse drug event with this |
| (Tolectin) | | of this medication. Initiate standard | normally. Prescribe with | medication due to decreased |
| | | recommended dose, but consider | standard precautions. | metabolism. Consider a reduction of |
| | | higher maintenance dose if indicated.* | | recommended starting dose and/or |
| | | | | reducing frequency of use. * |
| Fenoprofen | UGT2B7 | n/a | This patient is predicted to | This patient is at risk of experiencing |
| (Nalfon) | | | metabolize this medication | an adverse drug event with this |
| | | | normally. Prescribe with | medication due to decreased |
| | | | standard precautions. | metabolism. Consider a reduction of |
| | | | | recommended starting dose and/or |
| | | | | reducing frequency of use. * |
| | CYP1A2 & | CYP2C9 | | |
| | CYP2C9 | B | C | D, C/D |
| Naproxen | CYP1A2: A | This patient may experience treatment | This patient is predicted to metabolize | This patient is predicted to metabolize |
| (Naprosyn, | | failure due to increased metabolism | this medication normally. Prescribe | this medication normally. Prescribe |
| Aleve) | | of this medication. Initiate standard | with standard precautions. | with standard precautions. |
| | | recommended dose, but consider | | |
| | | higher maintenance dose if indicated. | | |
| | | * | | |
| | CYP1A2: | This patient is predicted to metabolize | This patient is predicted to metabolize | This patient is at risk of experiencing |
| | A/B, B | this medication normally. Prescribe | this medication normally. Prescribe | an adverse drug event with this |
| | | with standard precautions. | with standard precautions. | medication due to decreased |
| | | | | metabolism. Consider a reduction of |
| | | | | recommended starting dose and/or |
| | | | | reducing frequency of use. ** |
| | CYP1A2: | This patient is at risk of experiencing | This patient is at risk of experiencing | This patient is at risk of experiencing |
| | C, D | an adverse drug event with this | an adverse drug event with this | an adverse drug event with this |
| | | medication due to decreased | medication due to decreased | medication due to decreased |
| | | metabolism. Consider a reduction of | metabolism. Consider a reduction of | metabolism. Consider a reduction of |
| | | recommended starting dose and/or | recommended starting dose and/or | recommended starting dose and/or |
| | | reducing frequency of use. ** | reducing frequency of use. ** | reducing frequency of use. ** |
| | CYP2C8 & | CYP2C9 | | |
| | CYP2C9 | B | C | D, C/D |
| Ibuprofen | CYP2C8: B | This patient is predicted to metabolize | This patient is predicted to metabolize | This patient is at risk of experiencing |
| (Advil, | | this medication normally. Prescribe | this medication normally. Prescribe | an adverse drug event with this |
| Motrin) | | with standard precautions. *** | with standard precautions. ** | medication due to decreased |
| | | | | metabolism. Consider a reduction of |
| | | | | recommended starting dose and/or |
| | | | | reducing frequency of use. ** |
| | CYP2C8: C | This patient is at risk of experiencing | This patient is at risk of experiencing | This patient is at risk of experiencing |
| | | an adverse drug event with this | an adverse drug event with this | an adverse drug event with this |
| | | medication due to decreased | medication due to decreased | medication due to decreased |
| | | metabolism. Consider a reduction of | metabolism. Consider a reduction of | metabolism. Consider a reduction of |
| | | recommended starting dose and/or | recommended starting dose and/or | recommended starting dose and/or |
| | | reducing frequency of use. ** | reducing frequency of use. *** | reducing frequency of use. *** |

TABLE 2C Benzodiazepine drug recommendations based on CYP grade

| | | Metabolizing | CYP Genetic Report | | | |
|---|---|---|---|---|---|---|
| Benzodiazepines | [symbol] | enzyme | A | B | C | D |
| Alprazolam | [symbol] | CYP3A4/5 | This patient may | This patient is predicted | This patient is at risk of | This patient is at risk of |
| (Xanax) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but consider | | recommended dose, but | recommended dose, but |
| | | | higher maintenance | | consider lower maintenance | consider lower maintenance |
| | | | dose if indicated. H * | | dose if indicated. L * | dose if indicated. L * |
| Bromazepam | [symbol] | CYP1A2 | A or A/B: | This patient is predicted | This patient is at risk of | This patient is at risk of |
| (Lexotanil) | | | This patient may | to metabolize this | experiencing an adverse | experiencing an adverse |
| | | | experience treatment | medication normally. | drug event with this | drug event with this |
| | | | failure due to increased | Prescribe with | medication due to | medication due to |
| | | | metabolism of this | standard precautions. | decreased metabolism. | decreased metabolism. |
| | | | medication. Initiate | | Initiate standard | Initiate standard |
| | | | standard recommended | | recommended dose, but | recommended dose, but |
| | | | dose, but consider | | consider lower maintenance | consider lower maintenance |
| | | | higher maintenance | | dose if indicated. L * | dose if indicated. L * |
| | | | dose if indicated. H * | | | |
| Clonazepam | [symbol] | CYP3A4 | This patient may | This patient is predicted | This patient is at risk of | This patient is at risk of |
| (Klonopin) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but consider | | recommended dose, but | recommended dose, but |
| | | | higher maintenance | | consider lower maintenance | consider lower maintenance |
| | | | dose if indicated. H * | | dose if indicated. L * | dose if indicated. L * |
| Diazepam | [symbol] | CYP2C19 | This patient may | This patient is predicted | This patient is at risk of | This patient is at risk of |
| (Valium) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but consider | | recommended dose, but | recommended dose, but |
| | | | higher maintenance | | consider lower maintenance | consider lower maintenance |
| | | | dose if indicated. H * | | dose if indicated. L, S* | dose if indicated. L, S* |
| Flurazepam | [symbol] | n/a | This patient is predicted | This patient is predicted | This patient is predicted | This patient is predicted |
| (Dalmane) | | | to metabolize this | to metabolize this | to metabolize this | to metabolize this |
| | | | medication normally. | medication normally. | medication normally. | medication normally. |
| | | | Prescribe with | Prescribe with | Prescribe with | Prescribe with |
| | | | standard precautions. | standard precautions. | standard precautions. | standard precautions. |
| Lorazepam | [symbol] | UGT2B15 | n/a | This patient is predicted | This patient is at risk of | This patient is at risk of |
| (Ativan) | | | | to metabolize this | experiencing an adverse | experiencing an adverse |
| | | | | medication normally. | drug event with this | drug event with this |
| | | | | Prescribe with | medication due to | medication due to |
| | | | | standard precautions. | decreased metabolism. | decreased metabolism. |
| | | | | | Initiate standard | Initiate standard |
| | | | | | recommended dose, but | recommended dose, but |
| | | | | | consider lower maintenance | consider lower maintenance |
| | | | | | dose if indicated. L * | dose if indicated. L * |
| | | Metabolizing | CYP Genetic Report | | | |
| Benzodiazepines | [symbol] | enzyme | A | B | C | D |
| Midazolam | [symbol] | CYP3A4 | This patient may | This patient is predicted | This patient is at risk of | This patient is at risk of |
| (Versed) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but consider | | recommended dose, but | recommended dose, but |
| | | | higher maintenance | | consider lower maintenance | consider lower maintenance |
| | | | dose if indicated. H * | | dose if indicated. L * | dose if indicated. L * |
| Oxazepam | [symbol] | UGT2B15 | n/a | This patient is predicted | This patient is at risk of | This patient is at risk of |
| (Serax) | | | | to metabolize this | experiencing an adverse | experiencing an adverse |
| | | | | medication normally. | drug event with this | drug event with this |
| | | | | Prescribe with | medication due to | medication due to |
| | | | | standard precautions. | decreased metabolism. | decreased metabolism. |
| | | | | | Initiate standard | Initiate standard |
| | | | | | recommended dose, but | recommended dose, but |
| | | | | | consider lower maintenance | consider lower maintenance |
| | | | | | dose if indicated. L * | dose if indicated. L * |
| Temazepam | [symbol] | CYP2C19 | This patient may | This patient is predicted | This patient is at risk of | This patient is at risk of |
| (Restoril) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but consider | | recommended dose, but | recommended dose, but |
| | | | higher maintenance | | consider lower maintenance | consider lower maintenance |
| | | | dose if indicated. H * | | dose if indicated. L * | dose if indicated. L * |
| Triazolam | [symbol] | CYP3A4/5 | This patient may | This patient is predicted | This patient is at risk of | This patient is at risk of |
| (Halcion) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but consider | | recommended dose, but | recommended dose, but |
| | | | higher maintenance | | consider lower maintenance | consider lower maintenance |
| | | | dose if indicated. H * | | dose if indicated. L * | dose if indicated. L * |

| | |
|---|---|
| Letter Codes: | |
| H | Standard dose may result in lower plasma levels of active drug, use higher dose if indicated |
| L | Standard dose may result in higher plasma levels of active drug, use lower dose if indicated |
| C | Complex genotype with mutations in multiple enzymes that metabolize this drug. See full report for details |
| S | Higher risk of side effects |
| D | Consider alternative medication |
| Pi | Prodrug-Risk of increased activation and side effects |
| Pd | Prodrug-Risk of decreased activation and compromised efficacy |
| X | Use of this drug is contraindicated in patients with this genotype |
| F | On FDA biomarker list |
| G | Dosing guidelines exist for this drug (CPIC, Dutch or FDA) |
| M | Therapeutic drug monitoring may be indicated |

D Psychiatric drug recommendations based on CYP grade

| | | CYP2D6, | | | | |
|---|---|---|---|---|---|---|
| | | CYP2C19 | CYP2D6: A | CYP2D6: B | CYP2D6: C | CYP2D6: D |
| Citalopram | [symbol] | CYP2C19: A | This patient may | This patient may | This patient may | This patient has a |
| (Celexa) | | | experience treatment | experience treatment | experience treatment | complex genotype |
| | | | failure due to increased | failure due to increased | failure due to increased | that may be associated |
| | | | metabolism of this | metabolism of this | metabolism of this | with abnormal drug |
| | | | medication. Consider | medication. Consider | medication. Consider | metabolism. Consider |
| | | | alternative medication | an alternative | an alternative | an alternative |
| | | | not predominantly | medication not | medication not | medication not |
| | | | [symbol] | [symbol] | [symbol] | [symbol] |
| | | CYP2C19: B | This patient may | This patient is predicted | This patient is at risk of | This patient is at risk of |
| | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| | | CYP2C19: C | This patient has a | This patient is at risk of | This patient has a complex | This patient has a complex |
| | | | complex genotype that | experiencing an adverse | be genotype that may be | be genotype that may be |
| | | | may be associated with | drug event with this | associated with abnormal | associated with abnormal |
| | | | abnormal drug | medication due to | drug metabolism. Consider | drug metabolism. Consider |
| | | | metabolism. Initiate | decreased metabolism. | lower dose if indicated and | lower dose if indicated and |
| | | | standard recommended | Initiate standard | be alert to | be alert to |
| | | | dose, but monitor | recommended | [symbol] | [symbol] |
| | | | [symbol] | [symbol] | | |
| | | CYP2C19: D | This patient has a | This patient is at risk of | This patient is at risk of | This patient is at risk of |
| | | | complex genotype that | experiencing an adverse | experiencing an adverse | experiencing an adverse |
| | | | may be associated with | drug event with this | drug event with this | drug event with this |
| | | | abnormal drug | medication due to | medication due to | medication due to |
| | | | metabolism. Consider | decreased metabolism. | decreased metabolism. | decreased metabolism. |
| | | | a 50% reduction | Consider a 50% | Consider a 50% | Consider a 50% |
| | | | of recommended | reduction of | reduction of | reduction of |
| | | | [symbol] | [symbol] | [symbol] | [symbol] |
| | | CYP2D6, | | | | |
| | | CYP2C19 | CYP2D6: A | CYP2D6: B | CYP2D6: C | CYP2D6: D |
| Escitalopram | [symbol] | CYP2C19: A | This patient may | This patient may | This patient may | This patient has a |
| (Lexapro) | | | experience treatment | experience treatment | experience treatment | complex genotype |
| | | | failure due to increased | failure due to increased | failure due to increased | that may be associated |
| | | | metabolism of this | metabolism of this | metabolism of this | with abnormal drug |
| | | | medication. Consider | medication. Consider | medication. Consider | metabolism. Consider |
| | | | alternative medication | an alternative | an alternative | an alternative |
| | | | not predominantly | medication not | medication not | medication not |
| | | | [symbol] | [symbol] | [symbol] | [symbol] |
| | | CYP2C19: B | This patient may | This patient is predicted | This patient is at risk of | This patient is at risk of |
| | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| | | CYP2C19: C | This patient has a | This patient is at risk of | This patient has a complex | This patient has a complex |
| | | | complex genotype | experiencing an adverse | genotype that may be | genotype that may be |
| | | | that may be associated | drug event with this | associated with abnormal | associated with abnormal |
| | | | with abnormal drug | medication due to | drug metabolism. Consider | drug metabolism. Consider |
| | | | metabolism. Initiate | decreased metabolism. | lower dose if indicated and | lower dose if indicated and |
| | | | standard recommended | Initiate standard | be alert to | be alert to |
| | | | dose, but monitor | recommended | [symbol] | [symbol] |
| | | | [symbol] | [symbol] | | |
| | | CYP2C19: D | This patient has a | This patient is at risk of | This patient is at risk of | This patient is at risk of |
| | | | complex genotype that | experiencing an adverse | experiencing an adverse | experiencing an adverse |
| | | | may be associated with | drug event with this | drug event with this | drug event with this |
| | | | abnormal drug | medication due to | medication due to | medication due to |
| | | | metabolism. Consider | decreased metabolism. | decreased metabolism. | decreased metabolism. |
| | | | a 50% reduction | Consider a 50% | Consider a 50% | Consider a 50% |
| | | | of recommended | reduction of | reduction of | reduction of |
| | | | [symbol] | [symbol] | [symbol] | [symbol] |

| | | | | |
|---|---|---|---|---|
| CYP2D6, | | | | |
| CYP2C19 | CYP2D6: A | CYP2D6: B | CYP2D6: C | CYP2D6: D |

| | | | | | |
|---|---|---|---|---|---|
| Fluoxetine | [symbol] | CYP2D6 | This patient may experience | This patient is predicted to | This patient is at risk of |
| (Prozac) | | (minor: | treatment failure due to | metabolize this medication | experiencing an adverse drug |
| | | CYP2C19, | increased metabolism of this | normally. Prescribe with | event with this medication due |
| | | CYP3A4) | medication. Initiate standard | standard precautions. | to decreased metabolism. |
| | | | recommended dose, but | Please note: according to the | Initiate standard recommended |
| | | | [symbol] | FDA Drug | [symbol] |
| Fluvoxamine | [symbol] | CYP2D6 | This patient may experience | This patient is predicted to | This patient is at risk of |
| (Luvox) | | | treatment failure due to | metabolize this medication | experiencing an adverse drug |
| | | | increased metabolism of this | normally. Prescribe with | event with this medication due |
| | | | medication. Initiate standard | standard precautions. | to decreased metabolism. |
| | | | recommended dose, but | | Initiate standard recommended |
| | | | [symbol] | | [symbol] |
| Paroxetine | [symbol] | CYP2D6 | This patient may experience | This patient is predicted to | This patient is at risk of |
| (Paxil) | | | treatment failure due to | metabolize this medication | experiencing an adverse drug |
| | | | increased metabolism of this | normally. Prescribe with | event with this medication due |
| | | | medication. Consider an | standard precautions. | to decreased metabolism. |
| | | | alternative medication not | | Initiate standard recommended |
| | | | [symbol] | | [symbol] |
| Sertraline | [symbol] | CYP2C19 | This patient may experience | This patient is predicted to | This patient is predicted to |
| (Zoloft) | | | treatment failure due to | metabolize this medication | metabolize this medication |
| | | | increased metabolism of this | normally. Prescribe with | normally. Prescribe with |
| | | | medication. Initiate standard | standard precautions. | standard precautions. |
| | | | recommended dose, but | | |
| | | | [symbol] | | |

| | | CYP1A2, | | | | |
|---|---|---|---|---|---|---|
| | | CYP2D6 | CYP2D6: A | CYP2D6:B | CYP2D6: C | CYP2D6: D, C/D |
| Duloxetine | [symbol] | CYP1A2: A | This patient may | This patient may | This patient has a | This patient has a |
| (Cymbalta) | | | experience treatment | experience treatment | complex genotype | complex genotype |
| | | | failure due to increased | failure due to increased | that may be associated | that may be associated |
| | | | metabolism of this | metabolism of this | with abnormal drug | with abnormal drug |
| | | | medication. Consider | medication. Initiate | metabolism. Initiate | metabolism. Initiate |
| | | | a higher dose if | standard recommended | standard recommended | standard recommended |
| | | | indicated, or select an | dose, but | dose, but monitor | dose, but monitor |
| | | | [symbol] | [symbol] | [symbol] | [symbol] |
| | | CYP1A2: | This patient may | This patient may | This patient has a | This patient has a |
| | | A/B | experience treatment | experience treatment | complex genotype | complex genotype |
| | | | failure due to increased | failure due to increased | that may be associated | that may be associated |
| | | | metabolism of this | metabolism of this | with abnormal drug | with abnormal drug |
| | | | medication. Initiate | medication. Initiate | metabolism. Initiate | metabolism. Initiate |
| | | | standard recommended | standard recommended | standard recommended | standard recommended |
| | | | dose, but | dose, but | dose, but monitor | dose, but monitor |
| | | | [symbol] | [symbol] | [symbol] | [symbol] |
| | | CYP1A2: B | This patient may | This patient is predicted | This patient is at risk of | This patient is at risk of |
| | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| | | CYP1A2: C | This patient has a | This patient is at risk of | This patient is at risk of | This patient is at risk of |
| | | | complex genotype that | experiencing an adverse | experiencing an adverse | experiencing an adverse |
| | | | may be associated with | drug event with this | drug event with this | drug event with this |
| | | | abnormal drug | medication due to | medication due to | medication due to |
| | | | metabolism. Initiate | decreased metabolism. | decreased metabolism. | decreased metabolism. |
| | | | standard recommended | Initiate standard | Consider a lower dose if | Consider a lower dose if |
| | | | dose, but monitor | recommended | [symbol] | [symbol] |
| | | | [symbol] | [symbol] | | |
| | | CYP1A2: D | This patient has a | This patient is at risk of | This patient is at risk of | This patient is at risk of |
| | | | complex genotype that | experiencing an adverse | experiencing an adverse | experiencing an adverse |
| | | | may be associated with | drug event with this | drug event with this | drug event with this |
| | | | abnormal drug | medication due to | medication due to | medication due to |
| | | | metabolism. Initiate | decreased metabolism. | decreased metabolism. | decreased metabolism. |
| | | | standard recommended | Initiate standard | Consider a lower dose if | Consider a lower dose if |
| | | | dose, but monitor | recommended | [symbol] | [symbol] |
| | | | [symbol] | [symbol] | | |
| Levomil- | [symbol] | CYP3A4 | This patient may | This patient is predicted | This patient is at risk of | This patient is at risk of |
| nacipran | | | experience treatment | to metabolize this | experiencing an adverse | experiencing an adverse |
| (Fetzima) | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| | | CYP2D6 | | | | |
| | | (major), | | | | |
| | | CYP2C19 | | | | |
| | | (minor) | CYP2D6: A | CYP2D6: B | CYP2D6: C | CYP2D6: D |
| Venlafaxine | [symbol] | CYP2C19: A | This patient may | This patient may | This patient has a | This patient has a |
| (Effexor) | | | experience treatment | experience treatment | complex genotype that | complex genotype that |
| | | | failure due to increased | failure due to increased | may be associated with | may be associated with |
| | | | metabolism of this | metabolism of this | abnormal drug | abnormal drug |
| | | | medication. Conside | medication. Initiate | metabolism. Initiate | metabolism. Initiate |
| | | | titrating to a maximum | standard recommended | standard recommended | standard recommended |
| | | | of 150% of the | [symbol] | dose, but monitor | dose, but monitor |
| | | | [symbol] | | [symbol] | [symbol] |
| | | CYP2C19: B | This patient may | This patient is predicted | This patient is at risk of | This patient is at risk of |
| | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Conside | standard precautions. | decreased metabolism. | decreased metabolism. |
| | | | titrating to a maximum | | Consider a reduction of | Consider a reduction of |
| | | | of 150% of the | | [symbol] | [symbol] |
| | | | [symbol] | | | |
| | | CYP2C19: C | This patient has a | This patient is at risk of | This patient is at risk of | This patient is at risk of |
| | | | complex genotype | experiencing an adverse | experiencing an adverse | experiencing an adverse |
| | | | that may be associated | drug event with this | drug event with this | drug event with this |
| | | | with abnormal drug | medication due to | medication due | medication due |
| | | | metabolism. Initiate | decreased metabolism. | to decreased metabolism. | to decreased metabolism. |
| | | | standard recommended | Initiate standard | Consider a reduction of | Consider a reduction of |
| | | | dose, but monitor | recommended | [symbol] | [symbol] |
| | | | [symbol] | [symbol] | | |
| | | CYP2C19: D | This patient has a | This patient is at risk of | This patient is at risk of | This patient is at risk of |
| | | | complex genotype | experiencing an adverse | experiencing an adverse | experiencing an adverse |
| | | | that may be associated | drug event with this | drug event with this | drug event with this |
| | | | with abnormal drug | medication due to | medication due | medication due |
| | | | metabolism. Initiate | decreased metabolism. | to decreased metabolism. | to decreased metabolism. |
| | | | standard recommended | Initiate standard | Consider a reduction of | Consider a reduction of |
| | | | dose, but monitor | recommended | [symbol] | [symbol] |
| | | | [symbol] | [symbol] | | |
| Desipramine | [symbol] | CYP2D6 | This patient may | This patient is predicted | This patient is at risk of | This patient is at risk of |
| (Norpramin) | | | experience treatment | to metabolize this | experiencing an adverse | experiencing an adverse |
| | | | failure due to increased | medication normally. | drug event with this | drug event with this |
| | | | metabolism of this | Prescribe with | medication due | medication due to |
| | | | medication. Avoid | standard precautions. | to decreased metabolism. | decreased metabolism. |
| | | | tricyclic use, If a | | Consider a reduction of | Avoid tricyclic |
| | | | tricyclic is warranted, | | [symbol] | use. If a tricyclic is |
| | | | [symbol] | | | [symbol] |
| Nortriptyline | [symbol] | CYP2D6 | This patient may | This patient is predicted | This patient is at risk of | This patient is at risk of |
| (Pamelor) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Avoid | standard precautions. | decreased metabolism. | decreased metabolism. |
| | | | tricyclic use, If a | | Consider 25% | Avoid tricyclic |
| | | | tricyclic is warranted, | | reduction of | use. If a tricyclic is |
| | | | [symbol] | | [symbol] | [symbol] |

Atypical Antidepressants

| | | | A | B | C | D |
|---|---|---|---|---|---|---|
| Bupropion | [symbol] | CYP2B6 | n/a | This patient is predicted | This patient may | This patient may |
| (Wellbutrin) | | | | to metabolize this | experience treatment | experience treatment |
| | | | | medication normally. | failure due to decreased | failure due to decreased |
| | | | | Prescribe with | metabolism to active | metabolism to active drug. |
| | | | | standard precautions and | drug. Consider higher | Consider higher dose and |
| | | | | consider therapeutic drug | dose and therapeutic | therapeutic drug |
| | | | | | drug monitoring to | monitoring to |
| | | | | | [symbol] | [symbol] |
| Mirtazapine | [symbol] | CYP2D6 | This patient may | This patient is predicted | This patient is at risk of | This patient is at risk of |
| (Remeron) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| Nefazodone | [symbol] | CYP2D6 | This patient may | This patient is predicted | This patient is at risk of | This patient is at risk of |
| (Serzone) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| Trazodone | [symbol] | CYP2D6 | This patient may | This patient is predicted | This patient is at risk of | This patient is at risk of |
| (Oleptro) | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| Vilazodone | [symbol] | CYP3A4 | This patient may | This patient is predicted | This patient is at risk of | This patient is at risk of |
| (Viibryd) | | (major) | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| Vortioxetine | [symbol] | CYP2D6 | This patient may | This patient is predicted | This patient is at risk of | This patient is at risk of |
| (Brintellix) | | (major) | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism, |
| | | | standard recommended | | Initiate standard | Consider a lower dose if |
| | | | dose, but | | recommended | [symbol] |
| | | | [symbol] | | [symbol] | |

Typical Antipsychotics

| | | CYP2D6 | | | | |
|---|---|---|---|---|---|---|
| | | (major), | | | | |
| | | CYP1A2 | CYP2D6: A | CYP2D6: B | CYP2D6: C | CYP2D6: D |
| Chlor- | [symbol] | CYP1A2 A | This patient may | This patient may | This patient is predicted | This patient is at risk |
| promazine | | | experience treatment | experience treatment | to metabolize this | of experiencing an |
| (Thorazine) | | | failure due to increased | failure due to increased | medication normally. | adverse drug event |
| | | | metabolism of this | metabolism of this | Prescribe with | with this medication |
| | | | medication. Initiate | medication. Initiate | standard precautions. | due to decreased |
| | | | standard recommended | standard recommended | | metabolism. Consider |
| | | | dose, but | dose, but | | a reduction of |
| | | | [symbol] | [symbol] | | [symbol] |
| | | CYP1A2 B | This patient may | This patient is predicted | This patient is at risk of | This patient is at risk |
| | | | experience treatment | to metabolize this | experiencing an adverse | of experiencing an |
| | | | failure due to increased | medication normally. | drug event with this | adverse drug event |
| | | | metabolism of this | Prescribe with | medication due to | with this medication |
| | | | medication. Initiate | standard precautions. | decreased metabolism. | due to decreased |
| | | | standard recommended | | Initiate standard | metabolism. Consider |
| | | | dose, but | | recommended | a reduction of |
| | | | [symbol] | | [symbol] | [symbol] |
| | | CYP1A2 | This patient may | This patient may | This patient is at risk of | This patient is at risk |
| | | A/B | experience treatment | experience treatment | experiencing an adverse | of experiencing an |
| | | | failure due to increased | failure due to increased | drug event with this to | adverse drug event |
| | | | metabolism of this | metabolism of this | decreased metabolism. | with this medication |
| | | | medication. Initiate | medication. Initiate | Initiate standard | due to decreased |
| | | | standard recommended | standard recommended | recommended | metabolism. Consider |
| | | | dose, but | dose, but | [symbol] | a reduction of |
| | | | [symbol] | [symbol] | | [symbol] |
| | | CYP1A2 C | This patient is predicted | This patient is at risk | This patient is at risk | This patient is at risk |
| | | | to metabolize this | of experiencing an | of experiencing an | of experiencing an |
| | | | medication normally. | adverse drug event | adverse drug event | adverse drug event |
| | | | Prescribe with | with this medication | with this medication | with this medication |
| | | | standard precautions. | due to decreased | due to decreased | due to decreased |
| | | | | metabolism, Initiate | metabolism. Consider | metabolism. Consider |
| | | | | standard recommended | a reduction of | a reduction of |
| | | | | [symbol] | [symbol] | [symbol] |
| | | CYP1A2 D | This patient is at risk | This patient is at risk | This patient is at risk | This patient is at risk |
| | | | of experiencing an | of experiencing an | of experiencing an | of experiencing an |
| | | | adverse drug event | adverse drug event | adverse drug event | adverse drug event |
| | | | with this medication | with this medication | with this medication | with this medication |
| | | | due to decreased | due to decreased | due to decreased | due to decreased |
| | | | metabolism. Consider | metabolism. Consider | metabolism. Consider | metabolism. Consider |
| | | | a reduction of | a reduction of | a reduction of | a reduction of |
| | | | [symbol] | [symbol] | [symbol] | [symbol] |
| | | CYP2D6 | | | | |
| | | (major), | | | | |
| | | CYP1A2 | CYP2D6: A | CYP2D6: B | CYP2D6: C | CYP2D6: D |
| Fluphenazine | [symbol] | CYP1A2 A | This patient may | This patient may | This patient is predicted | This patient is at risk |
| (Prolixin) | | | experience treatment | experience treatment | to metabolize this | of experiencing an |
| | | | failure due to increased | failure due to increased | medication normally. | adverse drug event |
| | | | metabolism of this | metabolism of this | Prescribe with | with this medication |
| | | | medication. Initiate | medication. Initiate | standard precautions. | due to decreased |
| | | | standard recommended | standard recommended | | metabolism. Consider |
| | | | dose, but | dose, but | | a reduction of |
| | | | [symbol] | [symbol] | | [symbol] |
| | | CYP1A2 B | This patient may | This patient is predicted | This patient is at risk of | This patient is at risk |
| | | | experience treatment | to metabolize this | experiencing an adverse | of experiencing an |
| | | | failure due to increased | medication normally. | drug event with this | adverse drug event |
| | | | metabolism of this | Prescribe with | medication due to | with this medication |
| | | | medication. Initiate | standard precautions. | decreased metabolism. | due to decreased |
| | | | standard recommended | | Initiate standard | metabolism. Consider |
| | | | dose, but | | recommended | a reduction of |
| | | | [symbol] | | [symbol] | [symbol] |
| | | CYP1A2 | This patient may | This patient may | This patient may be at | This patient is at risk |
| | | A/B | experience treatment | experience treatment | risk of experiencing an | of experiencing an |
| | | | failure due to increased | failure due to increased | adverse drug event with | adverse drug event |
| | | | metabolism of this | metabolism of this | this medication due to | with this medication |
| | | | medication. Initiate | medication. Initiate | decreased metabolism. | due to decreased |
| | | | standard recommended | standard recommended | Initiate standard | metabolism. Consider |
| | | | dose, but | dose, but | recommended | a reduction of |
| | | | [symbol] | [symbol] | [symbol] | [symbol] |
| | | CYP1A2 C | This patient is predicted | This patient is at risk of | This patient is at risk | This patient is at risk |
| | | | to metabolize this | experiencing an adverse | of experiencing an | of experiencing an |
| | | | medication normally. | drug event with this | adverse drug event | adverse drug event |
| | | | Prescribe with | medication due to | with this medication | with this medication |
| | | | standard precautions. | decreased metabolism. | due to decreased | due to decreased |
| | | | | Initiate standard | metabolism. Consider | metabolism. Consider |
| | | | | recommended | a reduction of | a reduction of |
| | | | | [symbol] | [symbol] | [symbol] |
| | | CYP1A2 D | This patient is at risk | This patient is at risk | This patient is at risk | This patient is at risk |
| | | | of experiencing an | of experiencing an | of experiencing an | of experiencing an |
| | | | adverse drug event | adverse drug event | adverse drug event | adverse drug event |
| | | | with this medication | with this medication | with this medication | with this medication |
| | | | due to decreased | due to decreased | due to decreased | due to decreased |
| | | | metabolism. Consider | metabolism. Consider | metabolism. Consider | metabolism. Consider |
| | | | a reduction of | a reduction of | a reduction of | a reduction of |
| | | | [symbol] | [symbol] | [symbol] | [symbol] |

| | | | |
|---|---|---|---|
| CYP2D6: A | CYP2D6: B | CYP2D6: C | CYP2D6: D |

| | | | | | | |
|---|---|---|---|---|---|---|
| Haloperidol | [symbol] | CYP2D6 | This patient may | This patient is predicted | This patient is at risk | This patient is at risk of |
| (Haldol) | | | experience treatment | to metabolize this | of experiencing an | experiencing an adverse |
| | | | failure due to increased | medication normally. | adverse drug event | drug event with this |
| | | | metabolism of this | Prescribe with | with this medication | medication due to |
| | | | medication. Be alert to | standard precautions. | due to decreased | decreased metabolism. |
| | | | decreased haloperidol | | metabolism. Consider | Consider a 50% |
| | | | plasma | | a reduction of | reduction of |
| | | | [symbol] | | [symbol] | [symbol] |
| Perphenazine | [symbol] | CYP2D6 | This patient may | This patient is predicted | This patient is at risk | This patient is at risk |
| (Trilafon) | | | experience treatment | to metabolize this | of experiencing an | of experiencing an |
| | | | failure due to increased | medication normally. | adverse drug event | adverse drug event |
| | | | metabolism of this | Prescribe with | with this medication | with this medication |
| | | | medication. Initiate | standard precautions. | due to decreased | due to decreased |
| | | | standard recommended | | metabolism. Consider | metabolism. Consider |
| | | | dose, but | | a reduction of | a reduction of |
| | | | [symbol] | | [symbol] | [symbol] |
| Thioridazine | [symbol] | CYP2D6 | This patient may | This patient is predicted | This medication is | This medication is |
| (Mellaril) | | | experience treatment | to metabolize this | contraindicated in | contraindicated in |
| | | | failure due to increased | medication normally. | patients with | patients with |
| | | | metabolism of this | Prescribe with | reduced CYP2D6 | reduced CYP2D6 |
| | | | medication. Initiate | standard precautions. | activity due to | activity due to |
| | | | standard recommended | | potentially fatal | potentially fatal |
| | | | dose, but | | side effects. X, D, | side effects. X, D, |
| | | | [symbol] | | F *** | F *** |
| Thiothixene | [symbol] | CYP1A2 | A or A/B: This patient | This patient is predicted | This patient is at risk | This patient is at risk |
| (Navane) | | | may experience | to metabolize this | of experiencing an | of experiencing an |
| | | | treatment failure due to | medication normally. | adverse drug event | adverse drug event |
| | | | increased metabolism | Prescribe with | with this medication | with this medication |
| | | | of this medication. | standard precautions. | due to decreased | due to decreased |
| | | | Initiate standard | | metabolism. | metabolism. Consider |
| | | | recommended dose, | | Initiate standard | a reduction of |
| | | | [symbol] | | recommended | [symbol] |
| | | | | | [symbol] | |

Atypical Antipsychotics

| | | CYP2D6, | | | | |
|---|---|---|---|---|---|---|
| | | CYP3A4 | CYP2D6: A | CYP2D6: B | CYP2D6: C | CYP2D6: D |
| Aripiprazole | [symbol] | CYP3A4: A | This patient may | This patient may | This patient is predicted | This patient is at risk |
| (Abilify) | | | experience treatment | experience treatment | to metabolize this | of experiencing an |
| | | | failure due to increased | failure due to increased | medication normally. | adverse drug event |
| | | | metabolism of this | metabolism of this | Prescribe with | with this medication |
| | | | medication. Consider a | medication. Initiate | standard precautions. | due to decreased |
| | | | higher dose if indicated, | standard recommended | | metabolism. Consider |
| | | | or select an | dose, but | | reducing maximum |
| | | | [symbol] | [symbol] | | [symbol] |
| | | CYP3A4: B | This patient may | This patient is predicted | This patient is at risk | This patient is at risk |
| | | | experience treatment | to metabolize this | of experiencing an | of experiencing an |
| | | | failure due to increased | medication normally. | adverse drug event | adverse drug event |
| | | | metabolism of this | Prescribe with | with this medication | with this medication |
| | | | medication. Initiate | standard precautions. | due to decreased | due to decreased |
| | | | standard recommended | | metabolism. | metabolism. Consider |
| | | | dose, but | | Initiate standard | reducing maximum |
| | | | [symbol] | | recommended | [symbol] |
| | | | | | [symbol] | |
| | | CYP3A4: C | This patient is predicted | This patient is at risk | This patient is at risk of | This patient is at risk |
| | | | to metabolize this | of experiencing an | experiencing an adverse | of experiencing an |
| | | | medication normally. | adverse drug event | drug event with this | adverse drug event |
| | | | Prescribe with | with this medication | medication due to | with this medication |
| | | | standard precautions. | due to decreased | decreased metabolism. | due to decreased |
| | | | | metabolism. | Consider a reduction of | metabolism. Consider |
| | | | | Initiate standard | [symbol] | reducing maximum |
| | | | | recommended | | [symbol] |
| | | | | [symbol] | | |
| | | CYP3A4: D | This patient is predicted | This patient is at risk | This patient is at risk of | This patient is at risk |
| | | | to metabolize this | of experiencing an | experiencing an adverse | of experiencing an |
| | | | medication normally. | adverse drug event | drug event with this | adverse drug event |
| | | | Prescribe with | with this medication | medication due to | with this medication |
| | | | standard precautions. | due to decreased | decreased metabolism. | due to decreased |
| | | | | metabolism. | Consider a reduction of | metabolism, Consider |
| | | | | Initiate standard | [symbol] | reducing maximum |
| | | | | recommended | | [symbol] |
| | | | | [symbol] | | |
| Asenapine | [symbol] | CYP1A2 | A or A/B: | This patient is predicted | This patient is at risk | This patient is at risk |
| (Saphris) | | | This patient may | to metabolize this | of experiencing an | of experiencing an |
| | | | experience treatment | medication normally. | adverse drug event | adverse drug event |
| | | | failure due to increased | Prescribe with | with this medication | with this medication |
| | | | metabolism of this | standard precautions. | due to decreased | due to decreased |
| | | | medication. Initiate | | metabolism. | metabolism. |
| | | | standard recommended | | Initiate standard | Initiate standard |
| | | | dose, | | recommended | recommended |
| | | | [symbol] | | [symbol] | [symbol] |
| | | CYP2D6, | | | | CYP2D6: D |
| | | CYP3A4 | CYP2D6: A | CYP2D6: B | CYP2D6: C | |
| Iloperidone | [symbol] | CYP3A4: A | This patient has | This patient may | This patient is predicted | This patient has a complex |
| (Fanapt) | | | potential for | experience treatment | to metabolize this | genotype with potential for |
| | | | significantly increased | failure due to increased | medication normally. | decreased metabolism of this |
| | | | clearance. Be alert to | metabolism of this | Prescribe with | medication. Consider a 50% |
| | | | treatment failure and | medication. Initiate | standard precautions. | reduction of recommended |
| | | | increase dose if | standard recommended | | [symbol] |
| | | | indicated or select | dose, but | | |
| | | | alternative | [symbol] | | |
| | | | [symbol] | | | |
| | | CYP3A4: B | This patient may | This patient is predicted | This patient is at risk of | This patient has a complex |
| | | | experience treatment | to metabolize this | experiencing an adverse | genotype with potential for |
| | | | failure due to increased | medication normally. | drug event with this | decreased metabolism of this |
| | | | metabolism of this | Prescribe with | medication due to | medication. Consider a 50% |
| | | | medication. Initiate | standard precautions. | decreased metabolism. | reduction of recommended |
| | | | standard recommended | | Consider a reduction of | [symbol] |
| | | | dose, but | | [symbol] | |
| | | | [symbol] | | | |
| | | CYP3A4: C | This patient is predicted | This patient is at risk | This patient is at risk of | This patient has a complex |
| | | | to metabolize this | of experiencing an | experiencing an adverse | genotype with potential for |
| | | | medication normally. | adverse drug event | drug event with this | decreased metabolism of this |
| | | | Prescribe with | with this medication | medication due to | medication. Consider a 50% |
| | | | standard precautions. | due to decreased | decreased metabolism. | reduction of recommended |
| | | | | metabolism. | Consider a reduction of | [symbol] |
| | | | | Initiate standard | [symbol] | |
| | | | | recommended | | |
| | | | | [symbol] | | |
| | | CYP3A4: D | This patient is predicted | This patient is at risk | This patient is at risk of | This patient has a complex |
| | | | to metabolize this | of experiencing an | experiencing an adverse | genotype with potential for |
| | | | medication normally. | adverse drug event | drug event with this | decreased metabolism of this |
| | | | Prescribe with | with this medication | medication due to | medication. Consider a 50% |
| | | | standard precautions. | due to decreased | decreased metabolism. | reduction of recommended |
| | | | | metabolism. | Consider a reduction of | [symbol] |
| | | | | Initiate standard | [symbol] | |
| | | | | recommended | | |
| | | | | [symbol] | | |
| Lurasidone | [symbol] | CYP3A4 | This patient may | This patient is predicted | This patient is at risk | This patient is at risk |
| (Latuda) | | | experience treatment | to metabolize this | of experiencing an | of experiencing an |
| | | | failure due to increased | medication normally. | adverse drug event | adverse drug event |
| | | | metabolism of this | Prescribe with | with this medication | with this medication |
| | | | medication. Initiate | standard precautions. | due to decreased | due to decreased |
| | | | standard recommended | | metabolism. | metabolism. |
| | | | dose, but | | Initiate standard | Initiate standard |
| | | | [symbol] | | recommended | recommended |
| | | | | | [symbol] | [symbol] |

| | | CYP1A2, | | | | |
|---|---|---|---|---|---|---|
| | | CYP2D6 | CYP2D6: A | CYP2D6: B | CYP2D6: C | CYP2D6: D |
| Olanzapine | [symbol] | CYP1A2 A | This patient may | This patient may | This patient is predicted | This patient is predicted |
| (Zyprexa) | | | experience treatment | experience treatment | to metabolize this | to metabolize this |
| | | | failure due to increased | failure due to increased | medication normally. | medication normally. |
| | | | metabolism of this | metabolism of this | Prescribe with | Prescribe with |
| | | | medication. Initiate | medication. Initiate | standard precautions. | standard precautions. |
| | | | standard recommended | standard recommended | | |
| | | | dose, but | dose, but | | |
| | | | [symbol] | [symbol] | | |
| | | CYP1A2 B | This patient may | This patient is predicted | This patient is at risk of | This patient is at risk of |
| | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Initiate standard |
| | | | dose, but | | recommended | recommended |
| | | | [symbol] | | [symbol] | [symbol] |
| | | CYP1A2 | This patient may | This patient may | This patient is at risk of | This patient is at risk of |
| | | A/B | experience treatment | experience treatment | experiencing an adverse | experiencing an adverse |
| | | | failure due to increased | failure due to increased | drug event with this | drug event with this |
| | | | metabolism of this | metabolism of this | medication due to | medication due to |
| | | | medication. Initiate | medication. Initiate | decreased metabolism. | decreased metabolism. |
| | | | standard recommended | standard recommended | Initiate standard | Initiate standard |
| | | | dose, but | dose, but | recommended | recommended |
| | | | [symbol] | [symbol] | [symbol] | [symbol] |
| | | CYP1A2 C | This patient is predicted | This patient is at risk of | This patient is at risk | This patient is at risk |
| | | | to metabolize this | experiencing an adverse | of experiencing an | of experiencing an |
| | | | medication normally. | drug event with this | adverse drug event | adverse drug event |
| | | | Prescribe with | medication due to | with this medication | with this medication |
| | | | standard precautions. | decreased metabolism. | due to decreased | due to decreased |
| | | | | Initiate standard | metabolism. Consider | metabolism. Consider |
| | | | | recommended | a reduction of | a reduction of |
| | | | | [symbol] | [symbol] | [symbol] |
| | | CYP1A2 D | This patient is predicted | This patient is at risk of | This patient is at risk | This patient is at risk |
| | | | to metabolize this | experiencing an adverse | of experiencing an | of experiencing an |
| | | | medication normally. | drug event with this | adverse drug event | adverse drug event |
| | | | Prescribe with | medication due to | with this medication | with this medication |
| | | | standard precautions. | decreased metabolism. | due to decreased | due to decreased |
| | | | | Initiate standard | metabolism. Consider | metabolism. Consider |
| | | | | recommended | a reduction of | a reduction of |
| | | | | [symbol] | [symbol] | [symbol] |
| Paliperidone | | CYP2D6 | This patient is predicted | This patient is predicted | This patient is predicted | This patient is predicted |
| (Invega) | | (minimally | to metabolize this | to metabolize this | to metabolize this | to metabolize this |
| | | metabolized) | medication normally. | medication normally. | medication normally. | medication normally. |
| | | | Prescribe with | Prescribe with | Prescribe with | Prescribe with |
| | | | standard precautions. | standard precautions. | standard precautions. | standard precautions. |
| | | CYP2D6, | | | | |
| | | CYP3A4 | | | | |
| | | and | CYP2D6: A | CYP2D6: B | CYP2D6: C | CYP2D6: D |
| Pimozide | [symbol] | CYP3A4: A | This patient may | This patient may | This patient is predicted | This patient is at risk of |
| (Orap) | | | experience treatment | experience treatment | to metabolize this | experiencing an adverse |
| | | | failure due to increased | failure due to increased | medication normally. | drug event with this |
| | | | metabolism of this | metabolism of this | Prescribe with | medication due to |
| | | | medication. Consider | medication. Initiate | standard precautions. | decreased metabolism. |
| | | | a higher dose if | standard recommended | | Doses should not exceed |
| | | | indicated, or select an | dose, but | | [symbol] |
| | | | [symbol] | [symbol] | | |
| | | CYP3A4: B | This patient may | This patient is predicted | This patient is at risk of | This patient is at risk of |
| | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Doses should not exceed |
| | | | dose, but | | recommended | [symbol] |
| | | | [symbol] | | [symbol] | |
| | | CYP3A4: C | This patient is predicted | This patient is at risk of | This patient is at risk of | This patient is at risk of |
| | | | to metabolize this | experiencing an adverse | experiencing an adverse | experiencing an adverse |
| | | | medication normally. | drug event with this | drug event with this | drug event with this |
| | | | Prescribe with | medication due to | medication due to | medication due to |
| | | | standard precautions. | decreased metabolism. | decreased metabolism. | decreased metabolism. |
| | | | | Initiate standard | Initiate standard | Consider an alternative |
| | | | | recommended | recommended | [symbol] |
| | | | | [symbol] | [symbol] | |
| | | CYP3A4: D | This patient is predicted | This patient is at risk of | This patient is at risk of | This patient is at risk of |
| | | | to metabolize this | experiencing an adverse | experiencing an adverse | experiencing an adverse |
| | | | medication normally. | drug event with this | drug event with this | drug event with this |
| | | | Prescribe with | medication due to | medication due to | medication due to |
| | | | standard precautions. | decreased metabolism. | decreased metabolism. | decreased metabolism. |
| | | | | Initiate standard | Consider a reduction of | Consider an alternative |
| | | | | recommended | [symbol] | [symbol] |
| | | | | [symbol] | | |

| | | CYP2D6, | | | | |
|---|---|---|---|---|---|---|
| | | CYP3A4 | | | | |
| | | and | CYP2D6: A | CYP2D6: B | CYP2D6: C | CYP2D6: D |
| Pimozide | [symbol] | CYP3A4: A | This patient may | This patient may | This patient is predicted | This patient is at risk of |
| (Orap) | | | experience treatment | experience treatment | to metabolize this | experiencing an adverse |
| | | | failure due to increased | failure due to increased | medication normally. | drug event with this |
| | | | metabolism of this | metabolism of this | Prescribe with | medication due to |
| | | | medication. Consider a | medication. Initiate | standard precautions. | decreased metabolism. |
| | | | higher dose if indicated, | standard recommended | | Doses should not exceed |
| | | | or select an | dose, but | | [symbol] |
| | | | [symbol] | [symbol] | | |
| | | CYP3A4: B | This patient may | This patient is predicted | This patient is at risk of | This patient is at risk of |
| | | | experience treatment | to metabolize this | experiencing an adverse | experiencing an adverse |
| | | | failure due to increased | medication normally. | drug event with this | drug event with this |
| | | | metabolism of this | Prescribe with | medication due to | medication due to |
| | | | medication. Initiate | standard precautions. | decreased metabolism. | decreased metabolism. |
| | | | standard recommended | | Initiate standard | Doses should not exceed |
| | | | dose, but | | recommended | [symbol] |
| | | | [symbol] | | [symbol] | |
| | | CYP3A4: C | This patient is predicted | This patient is at risk of | This patient is at risk of | This patient is at risk of |
| | | | to metabolize this | experiencing an adverse | experiencing an adverse | experiencing an adverse |
| | | | medication normally. | drug event with this | drug event with this | drug event with this |
| | | | Prescribe with | medication due to | medication due to | medication due to |
| | | | standard precautions. | decreased metabolism. | decreased metabolism. | decreased metabolism, |
| | | | | Initiate standard | Initiate standard | Consider an alternative |
| | | | | recommended | recommended | [symbol] |
| | | | | [symbol] | [symbol] | |
| | | CYP3A4: D | This patient is predicted | This patient is at risk of | This patient is at risk of | This patient is at risk of |
| | | | to metabolize this | experiencing an adverse | experiencing an adverse | experiencing an adverse |
| | | | medication normally. | drug event with this | drug event with this | drug event with this |
| | | | Prescribe with | medication due to | medication due to | medication due to |
| | | | standard precautions. | decreased metabolism. | decreased metabolism. | decreased metabolism. |
| | | | | Initiate standard | Consider an alternative | Consider an alternative |
| | | | | recommended | [symbol] | [symbol] |
| | | | | [symbol] | | |

E Cardiology drug recommendations based on CYP grade

| | | | Metaboliz- | | | | |
|---|---|---|---|---|---|---|---|
| | | [symbol] | ing | CYP450 Genetic Report GRADE | | | |
| Drug Class | Drug | [symbol] | enzyme | A | B | C | D |
| Anti- | Clopidogrel | [symbol] | CYP2C19 | This patient is an | This patient is | This patient has | This patient has |
| platelets | (Plavix) | | | accelerated metabolizer | predicted to | decreased metabolism | decreased metabolism |
| | | | | of Clopidogrel into its | metabolize this | of Clopidogrel into its | of Clopidogrel into its |
| | | | | active form and may | medication normally. | active form and may | active form and may |
| | | | | therefore exhibit an | Prescribe with | therefore experience | therefore experience |
| | | | | increased antiplatelet | standard precautions. | reduced effectiveness | reduced effectiveness |
| | | | | response to standard | | of this medication. | of this medication. |
| | | | | doses. Consider | | Consider alternative | Consider alternative |
| | | | | lowering the dose or | | antiplatelet therapy to | antiplatelet therapy to |
| | | | | select an alternative | | mitigate risk of adverse | mitigate risk of adverse |
| | | | | medication to mitigate | | cardiovascular events. | cardiovascular events. |
| | | | | risk of adverse events. | | G, P, D, F | G, P, D, F, X |
| | | | | G, S, F | | | |
| | Prasugrel | [symbol] | CYP3A4, | CYP3A4: A | CYP3A4: B | CYP3A4: C | CYP3A4: D |
| | (Effient) | | CYP2B6 | | | | |
| | | | (minor | | | | |
| | | | CYP2C9, | | | | |
| | | | CYP266: B | This patient is predicted | This patient is | This patient is | This patient is predicted |
| | | | | to have accelerated | predicted to | predicted to | to have decreased |
| | | | | activation of Prasugrel | metabolize this | metabolize this | activation of Prasugrel |
| | | | | into its active metabolite. | medication normally. | medication normally. | into its active |
| | | | | Although the clinical | Prescribe with | Prescribe with | metabolite. Although |
| | | | | implications of increased | standard precautions. | standard precautions. | the clinical implications |
| | | | | metabolism for this | | | of decreased |
| | | | | medication are unclear, | | | metabolism for this |
| | | | | be alert to the potential | | | medication are unclear, |
| | | | | for adverse drug events. | | | be alert to the potential |
| | | | | P, D, F | | | for reduced platelet |
| | | | | | | | inhibition and consider |
| | | | | | | | alternative antiplatelet |
| | | | | | | | therapy. P, D, F |
| | | | CYP266: | This patient is predicted | This patient is | This patient is predicted | This patient is predicted |
| | | | C/D | to metabolize this | predicted to | to have decreased | to have decreased |
| | | | | medication normally. | metabolize this | activation of Prasugrel | activation of Prasugrel |
| | | | | Prescribe with | medication normally. | into its active metabolite. | into its active |
| | | | | standard precautions. | Prescribe with | Although the clinical | metabolite. Although |
| | | | | | standard precautions. | implications of decreased | the clinical implications |
| | | | | | | metabolism for this | of decreased |
| | | | | | | medication are unclear, | metabolism for this |
| | | | | | | be alert to the potential | medication are unclear, |
| | | | | | | for reduced platelet | be alert to the potential |
| | | | | | | inhibition and consider | for reduced platelet |
| | | | | | | alternative antiplatelet | inhibition and consider |
| | | | | | | therapy. P, D, F | alternative antiplatelet |
| | | | | | | | therapy. P, D, F |
| | Ticagrelor | [symbol] | CYP3A4/5 | This patient may | This patient is | This patient is at risk of | This patient is at risk of |
| | (Brilinta) | | | experience reduced | predicted to | experiencing an adverse | experiencing an adverse |
| | | | | efficacy of this | metabolize this | drug event with this | drug event with this |
| | | | | medication due to | medication normally. | medication due to | medication due to |
| | | | | accelerated metabolism. | Prescribe with | decreased metabolism. | decreased metabolism. |
| | | | | Initiate standard | standard precautions. | Initiate standard | Initiate standard |
| | | | | recommended dose, | | recommended dose, | recommended dose, |
| | | | | but consider higher | | but consider lower | but consider lower |
| | | | | maintenance | | maintenance dose | maintenance dose |
| | | | | dose if indicated. H | | if indicated, L, S, F | if indicated, L, S, F |
| | Vorapaxar | [symbol] | CYP3A4 | This patient may | This patient is | This patient is at risk of | This patient is at risk of |
| | (Zontivity) | | | experience reduced | predicted to | experiencing an adverse | experiencing an adverse |
| | | | | efficacy of this | metabolize this | drug event with this | drug event with this |
| | | | | medication due to | medication normally. | medication due to | medication due to |
| | | | | accelerated metabolism. | Prescribe with | decreased metabolism. | decreased metabolism. |
| | | | | Initiate standard | standard precautions. | Initiate standard | Initiate standard |
| | | | | recommended dose, | | recommended dose, | recommended dose, |
| | | | | but consider higher | | but consider lower | but consider lower |
| | | | | maintenance | | maintenance dose | maintenance dose |
| | | | | dose if indicated. H | | if indicated, L, S | if indicated, L, S |
| Anti- | Dabigatran | [symbol] | UGT2B15, | This patient is | This patient is | This patient is | This patient is |
| coagulants | (Pradaxa) | | UGT1A9, | predicted to | predicted to | predicted to | predicted to |
| | | | UGT2B7 | metabolize this | metabolize this | metabolize this | metabolize this |
| | | | | medication normally. | medication normally. | medication normally. | medication normally. |
| | | | | Prescribe with | Prescribe with | Prescribe with | Prescribe with |
| | | | | standard precautions. | standard precautions. | standard precautions. | standard precautions. |
| | Rivaroxaban | [symbol] | CYP3A4/5 | This patient may | This patient is | This patient is at risk | This patient is at risk |
| | (Xarelto) | | | experience reduced | predicted to | of experiencing an | of experiencing an |
| | | | | efficacy of this | metabolize this | adverse drug event | adverse drug event |
| | | | | medication due to | medication normally. | with this medication | with this medication |
| | | | | accelerated metabolism. | Prescribe with | due to decreased | due to decreased |
| | | | | Initiate standard | standard precautions. | metabolism. Initiate | metabolism. Initiate |
| | | | | recommended dose, | | standard recommended | standard recommended |
| | | | | but consider | | dose, but consider | dose, but consider |
| | | | | higher maintenance | | lower maintenance | lower maintenance |
| | | | | dose if indicated. H | | dose if indicated. L | dose if indicated. L |
| | Warfarin | | CYP2C9 | See separate “Warfarin” tab for logic | | | |
| | (Coumadin) | | and | | | | |
| | | | VKORC1 | | | | |
| Angiotensin | Losartan | [symbol] | CYP2C9 | n/a | This patient is | This patient may | This patient may |
| II Receptor | (Cozaar) | | | | predicted to | experience decreased | experience decreased |
| Blockers | | | | | metabolize this | antihypertensive | antihypertensive |
| | | | | | medication normally. | efficacy of this | efficacy of this |
| | | | | | Prescribe with | medication due to | medication due to |
| | | | | | standard precautions. | decreased conversion of | minimal conversion of |
| | | | | | | Losartan into its more | Losartan into its more |
| | | | | | | active form. Consider | active form. Consider |
| | | | | | | alternative medication | alternative medication |
| | | | | | | if indicated. P, H, D | if indicated. P, H, D |
| | Irbesartan | [symbol] | CYP2C9 | n/a | This patient is | This patient is at risk of | This patient is at risk of |
| | (Avapro) | | | | predicted to | experiencing an adverse | experiencing an adverse |
| | | | | | metabolize this | drug event with this | drug event with this |
| | | | | | medication normally. | medication due to | medication due to |
| | | | | | Prescribe with | decreased metabolism. | decreased metabolism. |
| | | | | | standard precautions. | Initiate standard | Initiate standard |
| | | | | | | recommended dose, | recommended dose, |
| | | | | | | but consider lower | but consider lower |
| | | | | | | maintenance dose | maintenance dose |
| | | | | | | if indicated. L | if indicated. L |
| | Olmesartan | [symbol] | Non-CYP | This patient is | This patient is | This patient is | This patient is |
| | (Benicar) | | mediated. | predicted to | predicted to | predicted to | predicted to |
| | | | Olmesartan | metabolize this | metabolize this | metabolize this | metabolize this |
| | | | medoxomil | medication normally. | medication normally. | medication normally. | medication normally. |
| | | | is an ester | Prescribe with | Prescribe with | Prescribe with | Prescribe with |
| | | | prodrug that | standard precautions. | standard precautions. | standard precautions. | standard precautions. |
| | | | is | | | | |
| | | | hydrolyzed/ | | | | |
| | | | bioactivated | | | | |
| | | | to | | | | |
| | | | olmesartan | | | | |
| | | | during | | | | |
| | | | absorption | | | | |
| | | | in | | | | |
| | | | [symbol] | | | | |
| | Valsartan | [symbol] | CYP2C9 | n/a | This patient is | This patient is | This patient is |
| | (Diovan) | | (minor), | | predicted to | predicted to | predicted to |
| | | | 80% | | metabolize this | metabolize this | experience decreased |
| | | | excreted | | medication normally. | medication normally. | metabolism of this |
| | | | unchanged | | Prescribe with | Prescribe with | medication. Initiate |
| | | | | | standard precautions. | standard precautions. | standard recommended |
| | | | | | | | dose, but consider |
| | | | | | | | lower maintenance |
| | | | | | | | dose if indicated. L |
| Alpha-2 | Guanfacine | [symbol] | CYP3A4 | This patient may | This patient is | This patient is at risk | This patient is at risk |
| agonists | (Tenex) | | | experience reduced | predicted to | of experiencing an | of experiencing an |
| | | | | efficacy of this | metabolize this | adverse drug event | adverse drug event |
| | | | | medication due to | medication normally. | with this medication | with this medication |
| | | | | accelerated metabolism. | Prescribe with | due to decreased | due to decreased |
| | | | | Initiate standard | standard precautions. | metabolism. Initiate | metabolism. Initiate |
| | | | | recommended dose, | | standard recommended | standard recommended |
| | | | | but consider an | | dose, but consider | dose, but consider |
| | | | | increased maintenance | | lower maintenance | lower maintenance |
| | | | | dose within the | | dose if indicated. L | dose if indicated. L |
| | | | | recommended dose | | | |
| | | | | range if indicated. H | | | |
| | | | | CYP3A4: A | CYP3A4: B | CYP3A4: C | CYP3A4: D |
| Sodium | Propafenone | [symbol] | CYP2D6: A | This patient is at risk of | This patient is | This patient has a | This patient has a |
| Channel | (Rythmol) | | | experiencing an adverse | predicted to | complex genotype that | complex genotype that |
| Blockers | | | | drug event with this | metabolize this | may be associated with | may be associated with |
| | | | | medication due to | medication normally. | abnormal drug | abnormal drug |
| | | | | increased metabolism. | Prescribe with | metabolism. Adjust | metabolism. Adjust |
| | | | | Consider an alternative | standard precautions. | dose in response to | dose in response to |
| | | | | medication not | | plasma concentration | plasma concentration |
| | | | | predominantly | | and record ECG, or | and record ECG, or |
| | | | | metabolized by | | select alternative | select alternative |
| | | | | CYP2D6. | | medication. | medication. |
| | | | CYP2D6: B | This patient is | This patient is | This patient is at risk of | This patient is at risk of |
| | | | | predicted to | predicted to | experiencing an adverse | experiencing an adverse |
| | | | | metabolize this | metabolize this | drug event with this | drug event with this |
| | | | | medication normally. | medication normally. | medication due to | medication due to |
| | | | | Prescribe with | Prescribe with | decreased metabolism. | decreased metabolism. |
| | | | | standard precautions. | standard precautions. | Adjust dose in response | Adjust dose in response |
| | | | | | | to plasma concentration | to plasma concentration |
| | | | | | | and record ECG, or | and record ECG, or |
| | | | | | | select alternative | select alternative |
| | | | | | | medication. | medication. |
| | | | CYP2D6: C | This patient has a | This patient is at risk | This patient is at risk of | This patient is at risk of |
| | | | | complex genotype that | of experiencing an | experiencing an adverse | experiencing an adverse |
| | | | | may be associated with | adverse drug event | drug event with this | drug event with this |
| | | | | abnormal drug | with this medication | medication due to | medication due to |
| | | | | metabolism. Adjust | due to decreased | decreased metabolism. | decreased metabolism. |
| | | | | dose in response to | metabolism. Adjust | Consider an alternative | Consider an alternative |
| | | | | plasma concentration | dose in response to | medication. | medication. |
| | | | | and record ECG, or | plasma concentration | | |
| | | | | select alternative | and record ECG, or | | |
| | | | | medication. | select alternative | | |
| | | | | | medication. | | |
| | | | CYP2D6: D | This patient has a | This patient is at risk | This patient is at risk of | This patient is at risk of |
| | | | | complex genotype that | of experiencing an | experiencing an adverse | experiencing an adverse |
| | | | | may be associated with | adverse drug event | drug event with this | drug event with this |
| | | | | abnormal drug | with this medication | medication due to | medication due to |
| | | | | metabolism. Adjust dose | due to decreased | decreased metabolism. | decreased metabolism. |
| | | | | in response to plasma | metabolism. Adjust | Consider an alternative | Consider an alternative |
| | | | | concentration and record | dose in response to | medication. | medication. |
| | | | | ECG, or select | plasma concentration | | |
| | | | | alternative medication. | and record ECG, or | | |
| | | | | | select alternative | | |
| | | | | | medication. | | |
| | | | CYP2D6: | This patient has a | This patient is at risk | This patient is at risk of | This patient is at risk of |
| | | | C/D | complex genotype that | of experiencing an | experiencing an adverse | experiencing an adverse |
| | | | | may be associated with | adverse drug event | drug event with this | drug event with this |
| | | | | abnormal drug | with this medication | medication due to | medication due to |
| | | | | metabolism. Adjust dose | due to decreased | decreased metabolism. | decreased metabolism. |
| | | | | in response to plasma | metabolism. Adjust | Consider an alternative | Consider an alternative |
| | | | | concentration and record | dose in response to | medication. | medication. |
| | | | | ECG, or select | plasma concentration | | |
| | | | | alternative medication. | and record ECG, or | | |
| | | | | | select alternative | | |
| | | | | | medication. | | |
| | | | CYP2D6: | This patient has a | This patient is at risk | This patient is at risk of | This patient is at risk of |
| | | | B/C/D | complex genotype that | of experiencing an | experiencing an adverse | experiencing an adverse |
| | | | | may be associated with | adverse drug event | drug event with this | drug event with this |
| | | | | abnormal drug | with this medication | medication due to | medication due to |
| | | | | metabolism. Adjust dose | due to decreased | decreased metabolism. | decreased metabolism. |
| | | | | in response to plasma | metabolism. Adjust | Adjust dose in response | Adjust dose in response |
| | | | | concentration and record | dose in response to | to plasma concentration | to plasma concentration |
| | | | | ECG, or select | plasma concentration | and record ECG, or | and record ECG, or |
| | | | | alternative medication. | and record ECG, or | select alternative | select alternative |
| | | | | | select alternative | medication. | medication. |
| | | | | | medication. | | |
| | | | CYP2D6: | This patient may be at | This patient is | This patient is at risk of | This patient is at risk of |
| | | | A/B | risk of experiencing an | predicted to | experiencing an adverse | experiencing an adverse |
| | | | | adverse drug event with | metabolize this | drug event with this | drug event with this |
| | | | | this medication due to | medication normally. | medication due to | medication due to |
| | | | | the potential of increased | Prescribe with | decreased metabolism. | decreased metabolism. |
| | | | | metabolism. Monitor | standard precautions. | Adjust dose in response | Adjust dose in response |
| | | | | closely and consider an | | to plasma concentration | to plasma concentration |
| | | | | alternative medication | | and record ECG, or | and record ECG, or |
| | | | | if indicated. | | select alternative | select alternative |
| | | | | | | medication. | medication. |
| | Flecainide | | CYP2D6 | This patient may | This patient is | This patient is at risk of | This patient is at risk of |
| | (Tambocor) | | | experience reduced | predicted to | experiencing an adverse | experiencing an adverse |
| | | | | efficacy of this | metabolize this | drug event with this | drug event with this |
| | | | | medication due to | medication normally. | medication due to | medication due to |
| | | | | accelerated metabolism. | Prescribe with | decreased metabolism. | decreased metabolism. |
| | | | | Initiate standard | standard precautions. | Consider 25% reduction | Consider 50% reduction |
| | | | | recommended dose, but | | of recommended | of recommended |
| | | | | consider adjusting dose | | starting dose, and utilize | starting dose, and |
| | | | | in response to ECG | | ECG and therapeutic | utilize ECG and |
| | | | | and therapeutic | | drug monitoring to | therapeutic drug |
| | | | | drug monitoring. | | guide dose adjustments. | monitoring to guide |
| | | | | | | | dose adjustments. |
| | Mexiletine | | CYP2D6 | This patient may | This patient is | This patient is at risk of | This patient is at risk of |
| | (Mexitil) | | | experience reduced | predicted to | experiencing an adverse | experiencing an adverse |
| | | | | efficacy of this | metabolize this | drug event with this | drug event with this |
| | | | | medication due to | medication normally. | medication due to | medication due to |
| | | | | accelerated metabolism. | Prescribe with | decreased metabolism. | decreased metabolism. |
| | | | | Initiate standard | standard precautions. | Consider reduction of | Consider reduction of |
| | | | | recommended dose, but | | recommended starting | recommended starting |
| | | | | consider adjusting dose | | dose, and utilize ECG | dose, and utilize ECG |
| | | | | in response to ECG | | and therapeutic drug | and therapeutic drug |
| | | | | and therapeutic | | monitoring to guide | monitoring to guide |
| | | | | drug monitoring. | | dose adjustments. | dose adjustments. |

| | | | | CYP3A4: A | CYP3A4: B |
|---|---|---|---|---|---|
| Sodium | Propafenone | [symbol] | CYP2D6: A | This patient is at risk of | This patient is predicted to |
| Channel | (Rythmol) | | | experiencing an adverse drug event | metabolize this medication |
| Blockers | | | | with this medication due to | normally. Prescribe with standard |
| | | | | increased metabolism. Consider an | precautions . |
| | | | | alternative medication not | |
| | | | | predominantly metabolized by | |
| | | | | CYP2D6. | |
| | | | CYP2D6: B | This patient is predicted to | This patient is predicted to |
| | | | | metabolize this medication normally. | metabolize this medication |
| | | | | Prescribe with standard precautions. | normally. Prescribe with standard |
| | | | | | precautions. |
| | | | CYP2D6: C | This patient has a complex | This patient is at risk of |
| | | | | genotype that may be associated | experiencing an adverse drug event |
| | | | | with abnormal drug metabolism. | with this medication due to |
| | | | | Adjust dose in response to plasma | decreased metabolism. Adjust |
| | | | | concentration and record ECG, or | dose in response to plasma |
| | | | | select alternative medication. | concentration and record ECG, or |
| | | | | | select alternative medication. |
| | | | CYP2D6: D | This patient has a complex | This patient is at risk of |
| | | | | genotype that may be associated | experiencing an adverse drug event |
| | | | | with abnormal drug metabolism. | with this medication due to |
| | | | | Adjust dose in response to plasma | decreased metabolism. Adjust |
| | | | | concentration and record ECG, or | dose in response to plasma |
| | | | | select alternative medication. | concentration and record ECG, or |
| | | | | | select alternative medication. |
| | | | CYP2D6: C/D | This patient has a complex | This patient is at risk of |
| | | | | genotype that may be associated | experiencing an adverse drug event |
| | | | | with abnormal drug metabolism. | with this medication due to |
| | | | | Adjust dose in response to plasma | decreased metabolism. Adjust |
| | | | | concentration and record ECG, or | dose in response to plasma |
| | | | | select alternative medication. | concentration and record ECG, or |
| | | | | | select alternative medication. |
| | | | CYP2D6: | This patient has a complex | This patient may be at risk of |
| | | | B/C/D | genotype that may be associated | experiencing an adverse drug event |
| | | | | with abnormal drug metabolism. | with this medication due to |
| | | | | Adjust dose in response to plasma | decreased metabolism. Adjust |
| | | | | concentration and record ECG, or | dose in response to plasma |
| | | | | select alternative medication. | concentration and record ECG, or |
| | | | | | select alternative medication. |
| | | | CYP2D6: | This patient may be at risk of | This patient is predicted to |
| | | | A/B | experiencing an adverse drug event | metabolize this medication |
| | | | | with this medication due to the | normally. Prescribe with standard |
| | | | | potential of increased metabolism. | precautions. |
| | | | | Monitor closely and consider an | |
| | | | | alternative medication if indicated. | |
| | Flecainide | | CYP2D6 | This patient may experience reduced | This patient is predicted to |
| | (Tambocor) | | | efficacy of this medication due to | metabolize this medication |
| | | | | accelerated metabolism. Initiate | normally. Prescribe with standard |
| | | | | standard recommended dose, but | precautions |
| | | | | consider adjusting dose in response | |
| | | | | to ECG and therapeutic drug | |
| | | | | monitoring. | |
| | Mexiletine | | CYP2D6 | This patient may experience reduced | This patient is predicted to |
| | (Mexitil) | | | efficacy of this medication due to | metabolize this medication |
| | | | | accelerated metabolism. Initiate | normally. Prescribe with standard |
| | | | | standard recommended dose, but | precautions |
| | | | | consider adjusting dose in response | |
| | | | | to ECG and therapeutic drug | |
| | | | | monitoring. | |

| CVP3A4: C | CVP3A4: D |
|---|---|
| This patient has a complex | This patient has a complex |
| genotype that may be associated | genotype that may be associated |
| with abnormal drug metabolism. | With abnormal drug metabolism. |
| Adjust dose in response to plasma | Adjust dose in response to |
| concentration and record ECG, or | plasma concentration and record |
| select alternative medication | ECG, or select alternative medication. |
| This patient is at risk of | This patient is at risk of |
| experiencing an adverse drug event | experiencing an adverse drug |
| with this medication due to | event with this medication due to |
| decreased metabolism. Adjust dose | decreased metabolism. Adjust |
| in response to plasma concentration | dose in response to plasma |
| and record ECG, or select | concentration and record ECG, or |
| alternative medication. | select alternative medication. |
| This patient is at risk of | This patient is at risk of |
| experiencing an adverse drug event | experiencing an adverse drug |
| with this medication due to | event with this medication due to |
| decreased metabolism. Consider an | decreased metabolism. Consider |
| alternative medication. | an alternative medication. |
| This patient is at risk of | This patient is at risk of |
| experiencing an adverse drug event | experiencing an adverse drug |
| with this medication due to | event with this medication due to |
| decreased metabolism. Consider an | decreased metabolism. Consider |
| alternative medication. | an alternative medication. |
| This patient is at risk of | This patient is at risk of |
| experiencing an adverse drug event | experiencing an adverse drug |
| with this medication due to | event with this medication due to |
| decreased metabolism. Consider an | decreased metabolism. Consider |
| alternative medication. | an alternative medication. |
| This patient is at risk of | This patient is at risk of |
| experiencing an adverse drug event | experiencing an adverse drug |
| with this medication due to | event with this medication due to |
| decreased metabolism. Adjust dose | decreased metabolism. Adjust |
| in response to plasma concentration | dose in response to plasma |
| and record ECG, or select | concentration and record ECG, or |
| alternative medication. | select alternative medication. |
| This patient is at risk of | This patient is at risk of |
| experiencing an adverse drug event | experiencing an adverse drug |
| with this medication due to | event with this medication due to |
| decreased metabolism. Adjust dose | decreased metabolism. Adjust |
| in response to plasma concentration | dose in response to plasma |
| and record ECG, or select | concentration and record ECG, or |
| alternative medication. | select alternative medication. |
| This patient is at risk of | This patient is at risk of |
| experiencing an adverse drug event | experiencing an adverse drug |
| with this medication due to | event with this medication due to |
| decreased metabolism. Consider | decreased metabolism. Consider |
| 25°/o reduction of recommended | 50°/o reduction of recommended |
| starting dose, and utilize ECG and | starting dose, and utilize ECG and |
| therapeutic drug monitoring to guide | therapeutic drug monitoring to |
| dose adjustments. | guide dose adjustments. |
| This patient is at risk of | This patient is at risk of |
| experiencing an adverse drug event | experiencing an adverse drug |
| with this medication due to | event with this medication due to |
| decreased metabolism. Consider | decreased metabolism. Consider |
| reduction of recommended starting | reduction of recommended |
| dose, and utilize ECG and | starting dose, and utilize ECG and |
| therapeutic drug monitoring to guide | therapeutic drug monitoring to |
| dose adjustments. | guide dose adjustments. |

| | | CYP2D6, | | | | |
|---|---|---|---|---|---|---|
| | | CYP1A2 | CYP1A2: A | CYP1A2: B | CYP1A2: C | CYP1A2: D |
| Propranolol | [symbol] | CYP2D6: A | This patient may | This patient is | This patient has a | This patient has a |
| (Inderal) | | | experience reduced | predicted to | complex genotype | complex genotype |
| | | | efficacy of this | metabolize this | that may be | that may be |
| | | | medication due to | medication normally. | associated with | associated with |
| | | | accelerated metabolism. | Prescribe with | abnormal drug | abnormal drug |
| | | | Initiate standard | standard precautions. | metabolism. Monitor | metabolism. Monitor |
| | | | recommended dose, | | patient and adjust | patient and adjust |
| | | | but consider | | dose as necessary, | dose as necessary, |
| | | | higher maintenance | | or select alternative | or select alternative |
| | | | dose if indicated. H | | medication. | medication. |
| | | CYP2D6: B | This patient is | This patient is | This patient is at risk | This patient is at risk |
| | | | predicted to | predicted to | of experiencing | of experiencing |
| | | | metabolize this | metabolize this | an adverse drug event | an adverse drug event |
| | | | medication normally. | medication normally. | with this medication | with this medication |
| | | | Prescribe with | Prescribe with | due to decreased | due to decreased |
| | | | standard precautions. | standard precautions. | metabolism. Consider | metabolism. Consider |
| | | | | | a reduction of | a reduction of |
| | | | | | recommended | recommended |
| | | | | | starting dose and/or | starting dose and/or |
| | | | | | reducing frequency | reducing frequency |
| | | | | | of use. | of use. |
| | | CYP2D6: C | This patient has a | This patient is at risk | This patient is at risk | This patient is at risk |
| | | | complex genotype | of experiencing | of experiencing | of experiencing |
| | | | that may be | an adverse drug event | an adverse drug event | an adverse drug event |
| | | | associated with | with this medication | with this medication | with this medication |
| | | | abnormal drug | due to decreased | due to decreased | due to decreased |
| | | | metabolism. Monitor | metabolism. Consider | metabolism. Consider | metabolism. Consider |
| | | | patient and adjust | a reduction of | a reduction of | a reduction of |
| | | | dose as necessary, | recommended | recommended | recommended |
| | | | or select alternative | starting dose and/or | starting dose and/or | starting dose and/or |
| | | | medication. | reducing frequency | reducing frequency | reducing frequency |
| | | | | of use. | of use. | of use. |
| | | CYP2D6: D | This patient has a | This patient is at risk | This patient is at risk | This patient is at risk |
| | | | complex genotype | of experiencing | of experiencing | of experiencing |
| | | | that may be | an adverse drug event | an adverse drug event | an adverse drug event |
| | | | associated with | with this medication | with this medication | with this medication |
| | | | abnormal drug | due to decreased | due to decreased | due to decreased |
| | | | metabolism. Monitor | metabolism. Consider | metabolism. Consider | metabolism. Consider |
| | | | patient and adjust | a reduction of | a reduction of | a reduction of |
| | | | dose as necessary, | recommended | recommended | recommended |
| | | | or select alternative | starting dose and/or | starting dose and/or | starting dose and/or |
| | | | medication. | reducing frequency | reducing frequency | reducing frequency |
| | | | | of use. | of use. | of use. |
| | | CYP2D6: | This patient has a | This patient is at risk | This patient is at risk | This patient is at risk |
| | | C/D | complex genotype | of experiencing | of experiencing | of experiencing |
| | | | that may be | an adverse drug event | an adverse drug event | an adverse drug event |
| | | | associated with | with this medication | with this medication | with this medication |
| | | | abnormal drug | due to decreased | due to decreased | due to decreased |
| | | | metabolism. Monitor | metabolism. Consider | metabolism. Consider | metabolism. Consider |
| | | | patient and adjust | a reduction of | reduction of | reduction of |
| | | | dose as necessary, | recommended | recommended | recommended |
| | | | or select alternative | starting dose and/or | starting dose, or | starting dose, or |
| | | | medication. | reducing frequency | choose an | choose an |
| | | | | of use. | alternative medication. | alternative medication. |
| | | CYP2D6: | This patient may | This patient is | This patient has a | This patient has a |
| | | A/B | experience decreased | predicted to | complex genotype | complex genotype |
| | | | efficacy of this | metabolize this | that may be | that may be |
| | | | medication due to | medication normally. | associated with | associated with |
| | | | increased metabolism. | Prescribe with | abnormal drug | abnormal drug |
| | | | Initiate standard | standard precautions. | metabolism. Monitor | metabolism. Monitor |
| | | | recommended dose, | | patient and adjust | patient and adjust |
| | | | but consider higher | | dose as necessary, | dose as necessary, |
| | | | maintenance dose if | | or select alternative | or select alternative |
| | | | indicated. H | | medication. | medication. |
| | | CYP2D6: | This patient has a | This patient may be at | This patient may be at | This patient may be at |
| | | B/C/D | complex genotype | risk of experiencing an | risk of experiencing an | risk of experiencing an |
| | | | that may be | adverse drug event with | adverse drug event with | adverse drug event with |
| | | | associated with | this medication due to | this medication due to | this medication due to |
| | | | abnormal drug | decreased metabolism. | decreased metabolism. | decreased metabolism. |
| | | | metabolism. Monitor | Consider a reduction of | Consider a reduction of | Consider a reduction of |
| | | | patient and adjust | recommended starting | recommended starting | recommended starting |
| | | | dose as necessary, | dose and/or reducing | dose and/or reducing | dose and/or reducing |
| | | | or select alternative | frequency of | frequency of | frequency of |
| | | | medication. | use. | use. | use. |

| | | CYP2D6, | | | | |
|---|---|---|---|---|---|---|
| | | CYP2C9 | CYP2C9: A | CYP2C9: B | CYP2C9: C | CYP2C9: D or C/D |
| Carvedilol | [symbol] | CYP2D6: A | N/A | This patient may experience | This patient has a | This patient has a |
| (Coreg) | | | | accelerated metabolism of this | complex genotype that | complex genotype that |
| | | | | medication. Carvedilol is | may be associated with | may be associated with |
| | | | | metabolized into 3 active | abnormal drug | abnormal drug |
| | | | | metabolites that exhibit equal | metabolism, but it is | metabolism, but it is |
| | | | | or more potent beta blocking | unclear whether a dose | unclear whether a dose |
| | | | | activity, but weaker | adjustment is necessary. | adjustment is necessary. |
| | | | | vasodilating activity. | Monitor the patient | Monitor the patient |
| | | | | It is unclear whether dose | carefully and adjust | carefully and adjust |
| | | | | changes are necessary; | dose if indicated. | dose if indicated. |
| | | | | therefore monitor the patient | | |
| | | | | closely and adjust dose as | | |
| | | | | indicated. | | |
| | | CYP2D6: B | This patient is | This patient is | This patient is | This patient is |
| | | | predicted to | predicted to | predicted to | predicted to |
| | | | metabolize this | metabolize this | metabolize this | metabolize this |
| | | | medication normally. | medication normally. | medication normally. | medication normally. |
| | | | Prescribe with | Prescribe with | Prescribe with | Prescribe with |
| | | | standard precautions. | standard precautions. | standard precautions. | standard precautions. |
| | | CYP2D6: C | N/A | This patient is at risk | This patient is at risk | This patient is at risk |
| | | | | of experiencing | of experiencing | of experiencing |
| | | | | an adverse drug event | an adverse drug event | an adverse drug event |
| | | | | with this medication | with this medication | with this medication |
| | | | | due to decreased | due to decreased | due to decreased |
| | | | | metabolism. Consider | metabolism. Consider | metabolism. Consider |
| | | | | a reduction of | a reduction of | a reduction of |
| | | | | recommended | recommended | recommended |
| | | | | starting dose and/or | starting dose and/or | starting dose and/or |
| | | | | reducing frequency | reducing frequency | reducing frequency |
| | | | | of use. | of use. | of use. |
| | | CYP2D6: D | N/A | This patient is at risk | This patient is at risk | This patient is at risk |
| | | | | of experiencing | of experiencing | of experiencing |
| | | | | an adverse drug event | an adverse drug event | an adverse drug event |
| | | | | with this medication | with this medication | with this medication |
| | | | | due to decreased | due to decreased | due to decreased |
| | | | | metabolism. Consider | metabolism. Consider | metabolism. Consider |
| | | | | a reduction of | a reduction of | a reduction of |
| | | | | recommended | recommended | recommended |
| | | | | starting dose and/or | starting dose and/or | starting dose and/or |
| | | | | reducing frequency | reducing frequency | reducing frequency |
| | | | | of use. | of use. | of use. |
| | | CYP2D6: | N/A | This patient is at risk | This patient is at risk | This patient is at risk |
| | | C/D | | of experiencing | of experiencing | of experiencing |
| | | | | an adverse drug event | an adverse drug event | an adverse drug event |
| | | | | with this medication | with this medication | with this medication |
| | | | | due to decreased | due to decreased | due to decreased |
| | | | | metabolism. Consider | metabolism. Consider | metabolism. Consider |
| | | | | a reduction of | a reduction of | a reduction of |
| | | | | recommended | recommended | recommended |
| | | | | starting dose and/or | starting dose and/or | starting dose and/or |
| | | | | reducing frequency | reducing frequency | reducing frequency |
| | | | | of use. | of use. | of use. |
| | | CYP2D6: | N/A | This patient may | This patient has a | This patient has a |
| | | A/B | | experience accelerated | complex genotype that | complex genotype that |
| | | | | metabolism of this | may be associated with | may be associated with |
| | | | | medication. Carvedilol is | abnormal drug | abnormal drug |
| | | | | metabolized into 3 active | metabolism, but it is | metabolism, but it is |
| | | | | metabolites that exhibit | unclear whether a dose | unclear whether a dose |
| | | | | equal or more potent beta | adjustment is necessary. | adjustment is necessary. |
| | | | | blocking activity, but weaker | Monitor the patient | Monitor the patient |
| | | | | vasodilating activity. It is | carefully and adjust | carefully and adjust |
| | | | | unclear whether dose changes | dose if indicated. | dose if indicated. |
| | | | | are necessary; therefore | | |
| | | | | monitor the patient closely and | | |
| | | | | adjust dose as indicated. | | |
| | | CYP2D6: | N/A | This patient is at risk | This patient is at risk | This patient is at risk |
| | | B/C/D | | of experiencing | of experiencing | of experiencing |
| | | | | an adverse drug event | an adverse drug event | an adverse drug event |
| | | | | with this medication | with this medication | with this medication |
| | | | | due to decreased | due to decreased | due to decreased |
| | | | | metabolism. Consider | metabolism. Consider | metabolism. Consider |
| | | | | a reduction of | a reduction of | a reduction of |
| | | | | recommended | recommended | recommended |
| | | | | starting dose and/or | starting dose and/or | starting dose and/or |
| | | | | reducing frequency | reducing frequency | reducing frequency |
| | | | | of use. | of use. | of use. |
| Metoprolol | [symbol] | CYP2D6 | This patient may | This patient is | This patient is at risk of | This patient is at risk of |
| (Lopressor) | | | experience treatment | predicted to | experiencing an adverse | experiencing an adverse |
| | | | failure due to | metabolize this | drug event with this | drug event with this |
| | | | increased metabolism | medication normally. | medication due to | medication due to |
| | | | of this medication. | Prescribe with | decreased metabolism. | decreased metabolism. |
| | | | Consider a higher | standard precautions. | Consider a reduction of | Consider a reduction of |
| | | | dose if indicated, or | | dose and titrate to | dose and titrate to |
| | | | select an alternative | | response, or choose an | response, or choose an |
| | | | medication. (8) G, H, | | alternative medication. | alternative medication. |
| | | | D, F | | (8) G, L, S, D, F | (8) G, L, S, D, F |
| Nebivolol | [symbol] | CYP2D6 | This patient may | This patient is | This patient is at risk | This patient is at risk |
| (Bystolic) | | | experience reduced | predicted to | of experiencing | of experiencing |
| | | | efficacy of this | metabolize this | an adverse drug event | an adverse drug event |
| | | | medication due to | medication normally. | with this medication | with this medication |
| | | | accelerated | Prescribe with | due to decreased | due to decreased |
| | | | metabolism. Initiate | standard precautions. | metabolism. Consider | metabolism. Consider |
| | | | standard | | a reduction of | a reduction of |
| | | | recommended dose, | | recommended | recommended |
| | | | but consider an | | starting dose and/or | starting dose and/or |
| | | | increased | | reducing frequency | reducing frequency |
| | | | maintenance dose | | of use. | of use. |
| | | | within the | | | |
| | | | recommended dose | | | |
| | | | range if indicated. H | | | |
| Atenolol | [symbol] | [symbol] | This patient is | This patient is | This patient is | This patient is |
| (Tenormin) | | metabolized, | predicted to | predicted to | predicted to | predicted to |
| | | renal | metabolize this | metabolize this | metabolize this | metabolize this |
| | | [symbol] | medication normally. | medication normally. | medication normally. | medication normally. |
| | | | Prescribe with | Prescribe with | Prescribe with | Prescribe with |
| | | | standard precautions. | standard precautions. | standard precautions. | standard precautions. |
| Labetalol | [symbol] | UGT1A1 | This patient is | This patient is | This patient is | This patient is |
| (Trandate) | | and 2B7 | predicted to | predicted to | predicted to | predicted to |
| | | | metabolize this | metabolize this | metabolize this | metabolize this |
| | | | medication normally. | medication normally. | medication normally. | medication normally. |
| | | | Prescribe with | Prescribe with | Prescribe with | Prescribe with |
| | | | standard precautions. | standard precautions. | standard precautions. | standard precautions. |
| Sotalol | [symbol] | [symbol] | This patient is | This patient is | This patient is | This patient is |
| (Betapace) | | metabolized, | predicted to | predicted to | predicted to | predicted to |
| | | renal | metabolize this | metabolize this | metabolize this | metabolize this |
| | | [symbol] | medication normally. | medication normally. | medication normally. | medication normally. |
| | | | Prescribe with | Prescribe with | Prescribe with | Prescribe with |
| | | | standard precautions. | standard precautions. | standard precautions. | standard precautions. |
| Bisoprolol | [symbol] | CYP2D6 (?) | This patient is | This patient is | This patient is | This patient is |
| (Zebeta) | | | predicted to | predicted to | predicted to | predicted to |
| | | | metabolize this | metabolize this | metabolize this | metabolize this |
| | | | medication normally. | medication normally. | medication normally. | medication normally. |
| | | | Prescribe with | Prescribe with | Prescribe with | Prescribe with |
| | | | standard precautions. | standard precautions. | standard precautions. | standard precautions. |

| | | | | | |
|---|---|---|---|---|---|
| Amlodipine | [symbol] | CYP3A4 | This patient may experience reduced | This patient is predicted to | This patient is at risk of |
| (Norvasc) | | | efficacy of this medication due to | metabolize this medication | experiencing an adverse drug event |
| | | | accelerated metabolism. Initiate | normally. Prescribe with standard | with this medication due to |
| | | | standard recommended dose, but | precautions. | decreased metabolism. Consider a |
| | | | consider an increased maintenance | | reduction of recommended starting |
| | | | dose within the recommended dose | | dose and/or reducing frequency of |
| | | | range if indicated. H | | use. |
| Felodipine | [symbol] | CYP3A4 | This patient may experience reduced | This patient is predicted to | This patient is at risk of |
| (Plendil) | | | efficacy of this medication due to | metabolize this medication | experiencing an adverse drug event |
| | | | accelerated metabolism. Initiate | normally. Prescribe with standard | with this medication due to |
| | | | standard recommended dose, but | precautions. | decreased metabolism. Consider a |
| | | | consider an increased maintenance | | reduction of recommended starting |
| | | | dose within the recommended dose | | dose and/or reducing frequency of |
| | | | range if indicated. H | | use. |
| Diltiazem | [symbol] | CYP3A4 | This patient may experience reduced | This patient is predicted to | This patient is at risk of |
| (Cardizem) | | | efficacy of this medication due to | metabolize this medication | experiencing an adverse drug event |
| | | | accelerated metabolism. Initiate | normally. Prescribe with standard | with this medication due to |
| | | | standard recommended dose, but | precautions. | decreased metabolism. Consider a |
| | | | consider an increased maintenance | | reduction of recommended starting |
| | | | dose within the recommended dose | | dose and/or reducing frequency of |
| | | | range if indicated. H | | use. |
| Nifedipine | [symbol] | CYP3A4 | This patient may experience reduced | This patient is predicted to | This patient is at risk of |
| (Adalat) | | | efficacy of this medication due to | metabolize this medication | experiencing an adverse drug event |
| | | | accelerated metabolism. Initiate | normally. Prescribe with standard | with this medication due to |
| | | | standard recommended dose, but | precautions. | decreased metabolism. Consider a |
| | | | consider an increased maintenance | | reduction of recommended starting |
| | | | dose within the recommended dose | | dose and/or reducing frequency of |
| | | | range if indicated. H | | use. |
| Verapamil | [symbol] | CYP3A4 | This patient may experience reduced | This patient is predicted to | This patient is at risk of |
| (Calan, | | | efficacy of this medication due to | metabolize this medication | experiencing an adverse drug event |
| Verelan) | | | accelerated metabolism. Initiate | normally. Prescribe with standard | with this medication due to |
| | | | standard recommended dose, but | precautions. | decreased metabolism. Consider a |
| | | | consider an increased maintenance | | reduction of recommended starting |
| | | | dose within the recommended dose | | dose and/or reducing frequency of |
| | | | range if indicated. H | | use. |
| Atorvastatin | [symbol] | CYP3A4 | This patient may experience reduced | This patient is predicted to | This patient is at risk of |
| (Lipitor) | | | efficacy of this medication due to | metabolize this medication | experiencing an adverse drug event |
| | | | accelerated metabolism. Initiate | normally. Prescribe with standard | with this medication due to |
| | | | standard recommended dose, but | precautions. | decreased metabolism. Consider a |
| | | | consider an increased maintenance | | reduction of recommended starting |
| | | | dose within the recommended dose | | dose and/or reducing frequency of |
| | | | range if indicated. H | | use. |
| Fluvastatin | [symbol] | CYP2C9 | n/a | This patient is predicted to | This patient is at risk of |
| (Lescol) | | | | metabolize this medication | experiencing an adverse drug event |
| | | | | normally. Prescribe with standard | with this medication due to |
| | | | | precautions | decreased metabolism. Consider a |
| | | | | | reduction of recommended starting |
| | | | | | dose and/or reducing frequency of |
| | | | | | use. |
| Lovastatin | [symbol] | CYP3A4 E | This patient may experience reduced | This patient is predicted to | This patient is at risk of |
| (Mevacor) | | | efficacy of this medication due to | metabolize this medication | experiencing an adverse drug event |
| | | | accelerated metabolism. Initiate | normally. Prescribe with standard | with this medication due to |
| | | | standard recommended dose, but | precautions. | decreased metabolism. Consider a |
| | | | consider an increased maintenance | | reduction of recommended starting |
| | | | dose within the recommended dose | | dose and/or reducing frequency of |
| | | | range if indicated. H | | use. |
| Pravastatin | [symbol] | not | This patient is predicted to | This patient is predicted to | This patient is predicted to |
| (Pravachol) | | metabolized, | metabolize this medication | metabolize this medication | metabolize this medication |
| | | excretion: | normally. Prescribe with standard | normally. Prescribe with standard | normally. Prescribe with standard |
| | | renal 20%, | precautions | precautions | precautions |
| | | [symbol] | | | |
| Rosuvastatin | [symbol] | CYP2C9 | n/a | This patient is predicted to | This patient is predicted to |
| (Crestor) | | (minor) | | metabolize this medication | metabolize this medication |
| | | | | normally. Prescribe with standard | normally. Prescribe with standard |
| | | | | precautions | precautions |
| Simvastatin | [symbol] | CYP3A4 | This patient may experience reduced | This patient is predicted to | This patient is at risk of |
| (Zocor) | | | efficacy of this medication due to | metabolize this medication | experiencing an adverse drug event |
| | | | accelerated metabolism. Initiate | normally. Prescribe with standard | with this medication due to |
| | | | standard recommended dose, but | precautions. | decreased metabolism. Consider a |
| | | | consider an increased maintenance | | reduction of recommended starting |
| | | | dose within the recommended dose | | dose and/or reducing frequency of |
| | | | range if indicated. H | | use. |

Warfarin

| VKORC1 alleles | rs9923231 | rs17708472 | rs9934438 | rs2359612 | rs7294 | | |
|---|---|---|---|---|---|---|---|
| *1 | C | G | G | G | C | | |
| *2 | T | G | A | A | C | | |
| *3 | C | G | G | G | T | | |
| *4 | C | A | G | G | C | | |
| | Allele Set | activity score | grade | | listA | listB | |
| VKORC1 | *1/*1 | 2 | b | CYP2C9 | *2 | *3 | |
| Allele Set | *1/*2 | 1.5 | c | Allele | *5 | *6 | |
| | *1/*3 | 2.5 | b | Set | *8 | | |
| | *1/*4 | 2.5 | b | | *11 | | |
| | *2/*2 | 1 | d | | *13 | | |
| | *2/*3 | 2 | b | | If 2C9 genotype is not in the listA or B, | | |
| | *2/*4 | 2 | b | | then ″Prescribe warfarin with standard precautio [symbol] | | |
| | *3/*3 | 3 | a | | | | |
| | *3/*4 | 3 | a | | | | |
| | *4/*4 | 3 | a | | | | |
| | | CYP2C9 Allele Set | | | | | |
| | | *1/*1 | *1/*(listA) | *1/*(listB) | *(listA)/*(listA) | *(listA)/*(listB) | *(listB)/*(listB) |
| VKORC1 | *1/*1 | high | high | average | average | average | low |
| Allele Set | *1/*2 | high | average | average | average | low | low |
| | *1/*3 | high | high | average | average | average | low |
| | *1/*4 | high | high | average | average | average | low |
| | *2/*2 | average | average | low | low | low | low |
| | *2/*3 | high | average | average | average | low | warfarin. |
| | *2/*4 | high | average | average | average | low | low |
| | *3/*3 | high | high | average | average | average | low |
| | *3/*4 | high | high | average | average | average | low |
| | *4/*4 | high | high | average | average | average | low |

[symbol]

indicates data missing or illegible when filed

Detection of point mutations or other types of the allelic variants disclosed herein, can be accomplished several ways known in the art, such as by molecular cloning of the specified allele and subsequent sequencing of that allele using techniques known in the art. Alternatively, the gene sequences can be amplified directly from a genomic DNA preparation from the DNA sample using PCR, and the sequence composition is determined from the amplified product. As described more fully below, numerous methods are available for analyzing a subject's DNA for mutations at a given genetic locus such as the gene of interest.

One such detection method is allele specific hybridization using probes overlapping the polymorphic region and having, for example, about 5, or alternatively 10, or alternatively 20, or alternatively 25, or alternatively 30 nucleotides around the polymorphic region. In another embodiment, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described, e.g., in Cronin et al. (1996) Human Mutation 7:244.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

If the polymorphic region is located in the coding region of the gene of interest, yet other methods than those described above can be used for determining the identity of the allelic variant according to methods known in the art.

The genotype information obtained from analyzing a sample of a patient's genetic material may be utilized, according to the principles of the invention, to predict whether a patient has a level of risk associated with poor opioid maintenance response. The risk may be associated with a side effect the patient may be susceptible to developing, an efficacy of the drug to the patient specifically or some combination thereof. The genotype information of the patient may be combined with demographic information about the patient as described above.

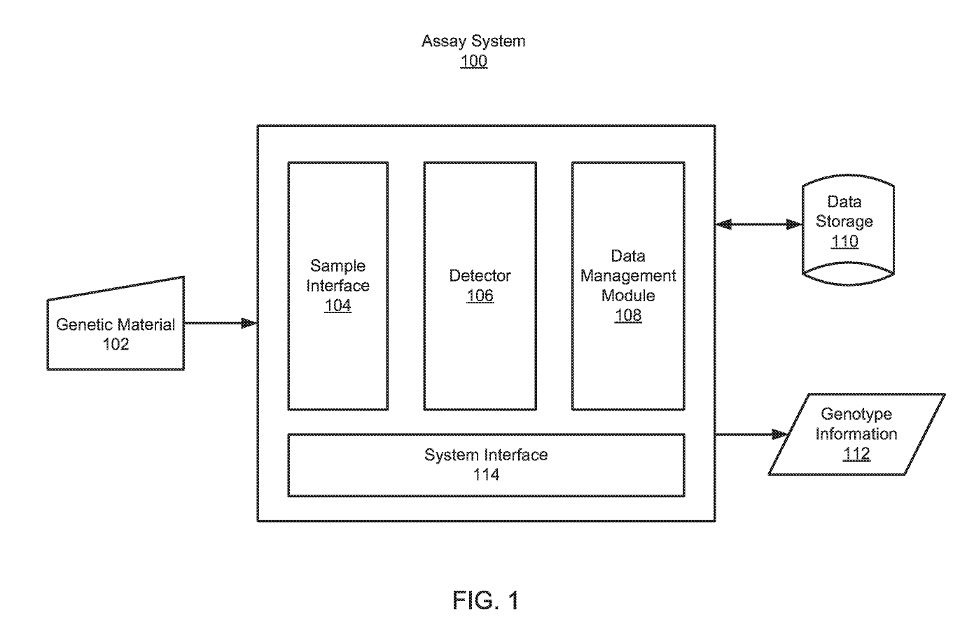

Referring to FIG. 1, depicted is an assay system 100. An assay system, such as assay system 100, may access or receive a genetic material, such as genetic material 102. The sample of genetic material 102 can be obtained from a patient by any suitable manner. The sample may be isolated from a source of a patient's DNA, such as saliva, buccal cells, hair roots, blood, cord blood, amniotic fluid, interstitial fluid, peritoneal fluid, chorionic villus, semen, or other suitable cell or tissue sample. Methods for isolating genomic DNA from various sources are well-known in the art. Also contemplated are non-invasive methods for obtaining and analyzing a sample of genetic material while still in situ within the patient's body.

The genetic material 102 may be received through a sample interface, such as sample interface 104 and detected using a detector, such as detector 106. A polymorphism may be detected in the sample by any suitable manner known in the art. For example, the polymorphism can be detected by techniques, such as allele specific hybridization, allele specific oligonucleotide ligation, primer extension, minisequencing, mass spectroscopy, heteroduplex analysis, single strand conformational polymorphism (SSCP), denaturing gradient gel electrophoresis (DGGE), oligonucleotide microarray analysis, temperature gradient gel electrophoresis (TGGE), and combinations thereof to produce an assay result. The assay result may be processed through a data management module, such as data management module 108, to produce genotype information 112. The genotype information 112 may include an assay result on whether the patients has a genotype including one or more of the allelic variants listed in Tables 1 and 3 above. The genotype information 112 may be stored in data storage 110 or transmitted to another system or entity via a system interface 114.

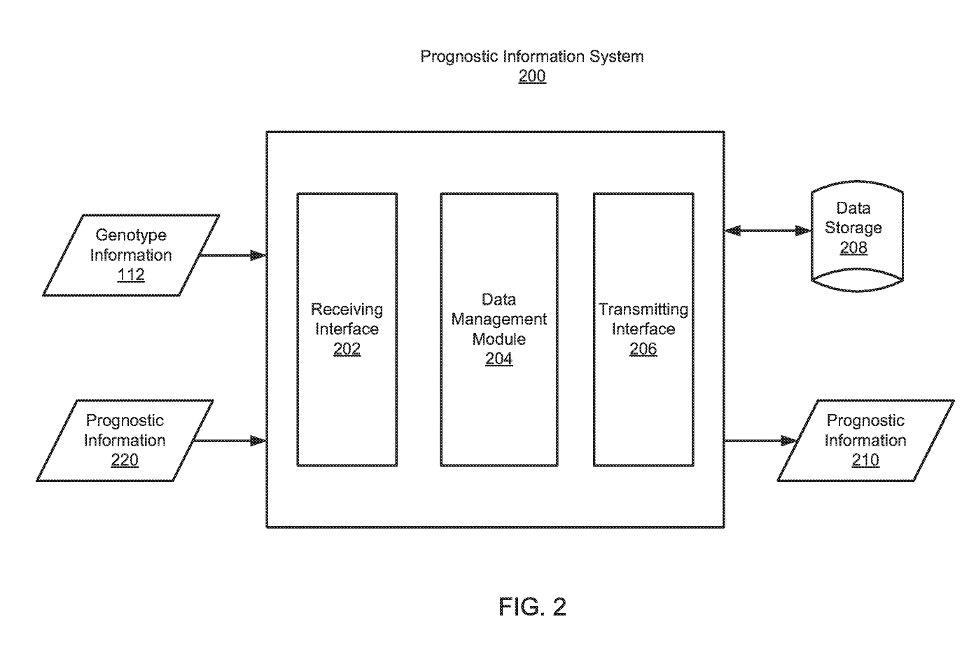

Referring to FIG. 2, depicted is a prognostic information system 200. The prognostic information system 200 may be remotely located away from the assay system 100 or operatively connected with it in an integrated system. The prognostic information system 200 receives the genotype information 112 through a receiving interface 202 for processing at a data management module 204 to generate prognostic information 210. The data management module 204 may utilize one or more algorithms described in greater detail below to generate prognostic information 210. The prognostic information 210 may be stored in data storage 208 or transmitted via a transmitting interface 206 to another system or entity. The transmitting interface 206 may be the same or different as the receiving interface 202. Furthermore, the system 200 may receive prognostic information 220 prepared by another system or entity. Prognostic information may be utilized, in addition to or in the alternative, to genotype information 112 in generating prognostic information 210.

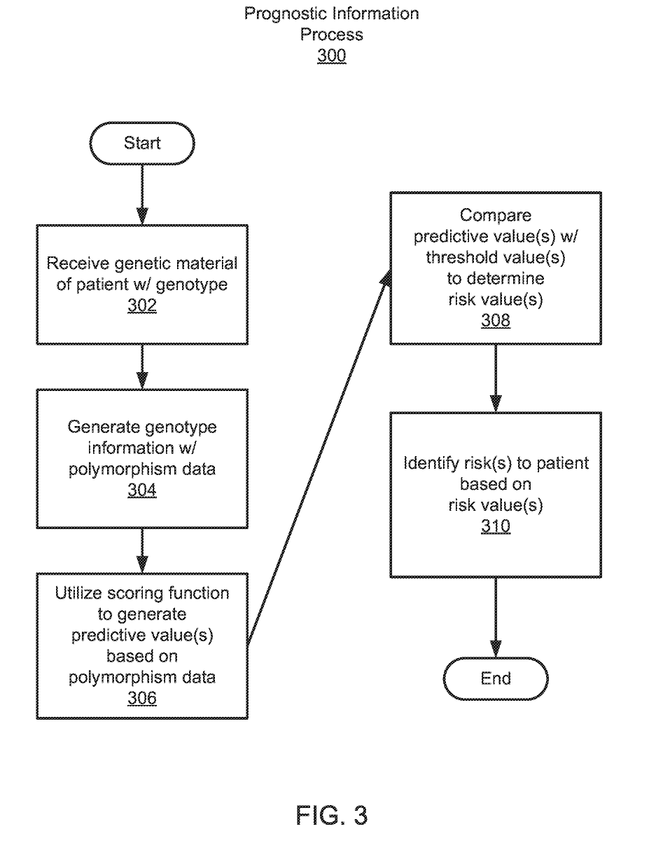

Referring to FIG. 3, depicted is a prognostic information process 300 which may be utilized for preparing information, such as genotype information 112 and prognostic information 210, utilizing an assay system, such as assay system 100 and/or a prognostic information system, such as prognostic information system 200, according to an embodiment. The steps of process 300, and other methods described herein, are described by way of example with the assay system 100 and the prognostic information system 200. The process 300 may be performed with other systems as well.

After process start, at step 302, a sample of genetic material of a patient is obtained as it is received at the sample interface 106. The sample interface can be any type of receptacle for holding or isolating the genetic material 102 for assay testing.

At step 304, the genetic material 102 is tested utilizing the detector 106 in assay system 100 to generate genotype information 112. The detector 106 may employ any of the assay methodologies described above to identify allelic variants in the genetic material 102 and generate the genotype information 112 including polymorphism data associated with one or more of the DNA polymorphisms described above in Tables 1 and 3. The data management module 108, utilizing a processor in an associated platform such as described below, may store the genotype information 112 on the data storage 110 and/or transmit the genotype information 112 to another entity or system, such as prognostic information system 200 where it is received at receiving interface 202 for analysis.

At step 306, the genotype information 112 can be analyzed utilizing a processor in an associated platform, such as described below, by using an algorithm which may be programmed for processing through data management module 204. The algorithm may utilize a scoring function to generate predictive values based on the polymorphism data in the genotype information 112. Different algorithms may be utilized to assign predictive values and aggregate values.

For example, an additive effect algorithm may be utilized to generate an analysis of a patient's genetic predisposition and their demographic phenotype predisposition to drug metabolism risk. In the additive effect algorithm, polymorphism data of the genotype information obtained from analyzing a patient's genetic material is utilized to indicate the active polymorphisms identified from a patient's genotype information. A tested polymorphism may be determined to be (1) absent or present in either (2) a heterozygous or (3) a homozygous variant in the patient's genotype. According to the additive effect algorithm, the polymorphisms identified from a patient's genotype information and demographic phenotype are each assigned a real value, such as an Odds Ratio (OR) or a parameter score, depending on which polymorphisms appears in the patient's genotype and demographic information.

To gather data for the algorithm, one or more of the alleles and haplotypes such as those listed in Table 1 may be tested and/or analyzed to produce one or more values associated with the presence or absence of the alleles and haplotypes. Other factors, such as other SNP Diploid Polymorphisms, other demographic phenotypes may also be tested and/or analyzed to produce one or more values associated with the presence or absence of the other SNP Diploid Polymorphisms and other demographic phenotypes.

The values gathered are based on results of the various tests and data gathered and/or determined. The values may be factored into an algorithm to score a subject's drug metabolism response based on the subject's genetic information and/or non-genetic characteristics or phenotypes. The algorithm may compute a composite score based on the results of individual tests. The composite score may be calculated based on an additive analysis of the individual scores which may be compared with a threshold value for determining drug metabolism response based on the additive score. In addition or in the alternative, more complex functions may be utilized to process the values developed from the testing results, such as utilizing one or more weighting factor(s) applied to one or more of the individual values based on various circumstances, such as if a subject was tested using specific equipment, a temporal condition, etc.

In all of the preceding examples, the predictive values and aggregate values generated are forms of prognostic information 210.

At step 310, the result of the comparison obtained in step 308 generates a second form of prognostic information 220. For example, (a) if the determined sum is higher than the threshold value, it can be predicted that the patient is at an elevated risk for drug metabolism risk associated with prescribing the patient a medication; (b) if the determined sum is at or near the threshold value, it can be predicted that the patient is at a moderate risk for drug metabolism risk; and (c) if the determined sum is below the threshold value, it can be predicted that the patient is at a low risk for drug metabolism risk.

Also at step 310, the data management module 205 in the prognostic information system 200 identifies a risk to a patient by executing an algorithm, such as the additive effect algorithm described above, and communicating the generated prognostic information 210. The data management module 204, utilizing a processor in an associated platform such as described below, may store the prognostic information 210 on the data storage 208 and/or transmit the prognostic information 210 to another entity or system prior to end of the prognostic information process 300. Other algorithms may also be used in a similar manner to generate useful forms of prognostic information for determining treatment options for a patient.

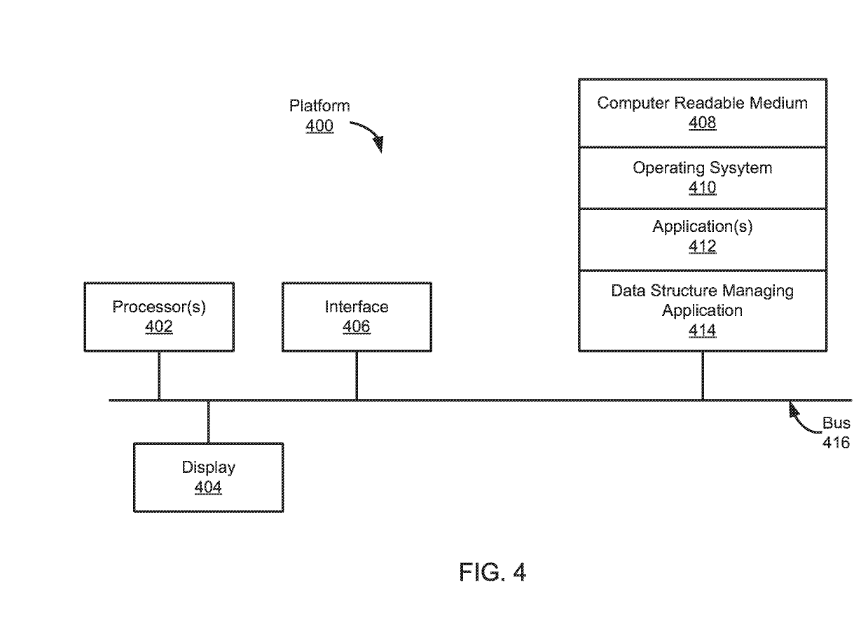

Referring to FIG. 4, there is shown a platform 400, which may be utilized as a computing device in a prognostic information system, such as prognostic information system 200, or an assay system, such as assay system 100. It is understood that the depiction of the platform 400 is a generalized illustration and that the platform 400 may include additional components and that some of the components described may be removed and/or modified without departing from a scope of the platform 400.

The platform 400 includes processor(s) 402, such as a central processing unit; a display 404, such as a monitor; an interface 406, such as a simple input interface and/or a network interface to a Local Area Network (LAN), a wireless 802.11x LAN, a 3G or 4G mobile WAN or a WiMax WAN; and a computer-readable medium (CRM) 408. Each of these components may be operatively coupled to a bus 416. For example, the bus 416 may be an EISA, a PCI, a USB, a FireWire, a NuBus, or a PDS.

A CRM, such as CRM 408 may be any suitable medium which participates in providing instructions to the processor(s) 402 for execution. For example, the CRM 408 may be non-volatile media, such as an optical or a magnetic disk; volatile media, such as memory; and transmission media, such as coaxial cables, copper wire, and fiber optics. Transmission media can also take the form of acoustic, light, or radio frequency waves. The CRM 408 may also store other instructions or instruction sets, including word processors, browsers, email, instant messaging, media players, and telephony code.

The CRM 408 may also store an operating system 410, such as MAC OS, MS WINDOWS, UNIX, or LINUX; application(s) 412, such as network applications, word processors, spreadsheet applications, browsers, email, instant messaging, media players such as games or mobile applications (e.g., “apps”); and a data structure managing application 414. The operating system 410 may be multi-user, multiprocessing, multitasking, multithreading, real-time and the like. The operating system 410 may also perform basic tasks such as recognizing input from the interface 406, including from input devices, such as a keyboard or a keypad; sending output to the display 404 and keeping track of files and directories on CRM 408; controlling peripheral devices, such as disk drives, printers, image capture devices; and for managing traffic on the bus 416. The applications 412 may include various components for establishing and maintaining network connections, such as code or instructions for implementing communication protocols including those such as TCP/IP, HTTP, Ethernet, USB, and FireWire.

A data structure managing application, such as data structure managing application 414 provides various code components for building/updating a computer-readable system architecture, such as for a non-volatile memory, as described above. In certain examples, some or all of the processes performed by the data structure managing application 412 may be integrated into the operating system 410. In certain examples, the processes may be at least partially implemented in digital electronic circuitry, in computer hardware, firmware, code, instruction sets, or any combination thereof.

Although described specifically throughout the entirety of the disclosure, the representative examples have utility over a wide range of applications, and the above discussion is not intended and should not be construed to be limiting. The terms, descriptions and figures used herein are set forth by way of illustration only and are not meant as limitations. Those skilled in the art recognize that many variations are possible within the spirit and scope of the principles of the invention. While the examples have been described with reference to the figures, those skilled in the art are able to make various modifications to the described examples without departing from the scope of the following claims, and their equivalents.

What is claimed is:

1. A method comprising facilitating a processing of and/or processing (1) data and/or (2) information and/or (3) at least one signal, the (1) data and/or (2) information and/or (3) at least one signal based, at least in part, on the following:
determining patient information, including DNA information, associated with a human subject;
determining from the DNA information whether a subject genotype of the human subject includes one or more alleles or haplotypes in at least one gene by detecting, utilizing a detection technology and the DNA information, a presence or absence of the one or more alleles or haplotypes in the one or more genes of the subject genotype,
wherein the at least one gene is selected from the gene group:
CYP2C8, CYP2C9, CYP2C19, CYP3A4, CYP3A5, CYP2D6, CYP1A2, VKORC1, UGT2B7, UGT2B15, CYP2B6 and CYP2E1;
determining at least one allele score associated with the alleles or haplotypes in the one or more genes based on the detected presence or absence of the one or more alleles or haplotypes in the one or more genes;
determining at least one grade associated with one or more genes of the at least one gene based on the determined at least one allele score; and
determining a drug metabolism response by the human subject associated with at least one drug based on the determined at least one grade associated with the one or more genes of the at least one gene.

2. A method of claim 1, wherein the (1) data and/or (2) information and/or (3) at least one signal are further based, at least in part, on the following:
determining at least one drug dosage recommendation based on the determined drug metabolism response associated with the at least one drug,
wherein the method for determining the drug metabolism response associated with the human subject, is an ex vivo method.

3. A method of claim 1, wherein the (1) data and/or (2) information and/or (3) at least one signal are further based, at least in part, on the following:
determining a comparing of a region, including the one or more alleles or haplotypes, of the subject genotype with a corresponding region of a predetermined reference genotype, wherein characteristics of the corresponding region of the reference genotype are based upon a predetermined population norm;
determining prognostic information associated with the human subject based on the determined drug metabolism response; and
determining a therapy for the human subject based on the determined prognostic information associated with the human subject.

4. A method of claim 1, wherein the at least one gene includes at least four genes selected from the gene group.

5. A method of claim 1, wherein the at least one gene includes at least eight genes selected from the gene group.

6. A method of claim 1, wherein the at least one gene includes at least nine genes selected from the gene group.

7. A method of claim 1, wherein the at least one gene includes at least eleven genes selected from the gene group.

8. An apparatus comprising:
at least one processor; and
at least one memory including computer program code for one or more programs,
the at least one memory and the computer program code configured to, with the at least one processor, cause the apparatus to perform at least the following,
determine patient information, including DNA information, associated with a human subject;
determine from the DNA information whether a subject genotype of the human subject includes one or more alleles or haplotypes in at least one gene by detecting, utilizing a detection technology and the DNA information, a presence or absence of the one or more alleles or haplotypes in the one or more genes of the subject genotype,
wherein the at least one gene is selected from the gene group:
CYP2C8, CYP2C9, CYP2C19, CYP3A4, CYP3A5, CYP2D6, CYP1A2, VKORC1, UGT2B7, UGT2B15, CYP2B6 and CYP2E1;
determine at least one allele score associated with the alleles or haplotypes in the one or more genes based on the detected presence or absence of the one or more alleles or haplotypes in the one or more genes;
determine at least one grade associated with one or more genes of the at least one gene based on the determined at least one allele score; and
determine a drug metabolism response by the human subject associated with at least one drug based on the determined at least one grade associated with the one or more genes of the at least one gene.

9. An apparatus of claim 8, wherein the (1) data and/or (2) information and/or (3) at least one signal are further based, at least in part, on the following:
determine at least one drug dosage recommendation based on the determined drug metabolism response associated with the at least one drug,
wherein the methodology associated with the apparatus for determining the drug metabolism response associated with the human subject, is an ex vivo methodology.

10. An apparatus of claim 8, wherein the apparatus is further caused to:
determine a comparing of a region, including the one or more alleles or haplotypes, of the subject genotype with a corresponding region of a predetermined reference genotype, wherein characteristics of the corresponding region of the reference genotype are based upon a predetermined population norm;
determine prognostic information associated with the human subject based on the determined drug metabolism response; and
determine a therapy for the human subject based on the determined prognostic information associated with the human subject.

11. An apparatus of claim 8, wherein the at least one gene includes at least four genes selected from the gene group.

12. An apparatus of claim 11, wherein the at least one gene includes at least eight genes selected from the gene group.

13. An apparatus of claim 8, wherein the at least one gene includes at least nine genes selected from the gene group.

14. An apparatus of claim 8, wherein the at least one gene includes at least eleven genes selected from the gene group.

15. A non-transitory computer readable medium storing computer readable instructions that when executed by at least one processor perform a method, the method comprising facilitating a processing of and/or processing (1) data and/or (2) information and/or (3) at least one signal, the (1) data and/or (2) information and/or (3) at least one signal based, at least in part, on the following:
determining patient information, including DNA information, associated with a human subject;
determining from the DNA information whether a subject genotype of the human subject includes one or more alleles or haplotypes in at least one gene by detecting, utilizing a detection technology and the DNA information, a presence or absence of the one or more alleles or haplotypes in the one or more genes of the subject genotype,
wherein the at least one gene is selected from the gene group:
CYP2C8, CYP2C9, CYP2C19, CYP3A4, CYP3A5, CYP2D6, CYP1A2, VKORC1, UGT2B7, UGT2B15, CYP2B6 and CYP2E1;
determining at least one allele score associated with the alleles or haplotypes in the one or more genes based on the detected presence or absence of the one or more alleles or haplotypes in the one or more genes;
determining at least one grade associated with one or more genes of the at least one gene based on the determined at least one allele score; and
determining a drug metabolism response by the human subject associated with at least one drug based on the determined at least one grade associated with the one or more genes of the at least one gene.

16. A computer readable medium of claim 15, wherein the (1) data and/or (2) information and/or (3) at least one signal are further based, at least in part, on the following:
determining at least one drug dosage recommendation based on the determined drug metabolism response associated with the at least one drug,
wherein the methodology associate with the medium for determining the drug metabolism response associated with the human subject, is an ex vivo methodology.

17. A computer readable medium of claim 15, wherein the (1) data and/or (2) information and/or (3) at least one signal are further based, at least in part, on the following:
determining a comparing of a region, including the one or more alleles or haplotypes, of the subject genotype with a corresponding region of a predetermined reference genotype, wherein characteristics of the corresponding region of the reference genotype are based upon a predetermined population norm;
determining prognostic information associated with the human subject based on the determined drug metabolism response; and
determining a therapy for the human subject based on the determined prognostic information associated with the human subject.

18. A computer readable medium of claim 15, wherein the at least one gene includes at least eight genes selected from the gene group.

19. A computer readable medium of claim 15, wherein the at least one gene includes at least nine genes selected from the gene group.

20. A computer readable medium of claim 15, wherein the at least one gene includes at least eleven genes selected from the gene group.
